(19) United States
(12) Patent Application Publication
(10) Pub. No.: US 2018/0015101 A1
(43) Pub. Date: Jan. 18, 2018

(54) COMPOSITIONS AND METHODS FOR ANTIGEN-SPECIFIC TOLERANCE

(71) Applicants: INSERM (INSTITUT NATIONAL DE LA SANTÉ ET DE LA RECHERCHE MÉDICALE), Paris (FR); UNIVERSITÉ DE NANTES, Nantes (FR); ONIRIS, Nantes (FR)

(72) Inventors: Ignacio ANEGON, Nantes (FR); Philippe BLANCOU, Valbonne (FR); Thomas SIMON, Baltimore, MD (US); Julien POGU, Nantes (FR)

(21) Appl. No.: 15/521,950

(22) PCT Filed: Oct. 27, 2015
(86) PCT No.: PCT/EP2015/074819

§ 371 Date: Aug. 10, 2017

(30) Foreign Application Priority Data
Oct. 28, 2014 (EP) 14306723.9

(51) Int. Cl. A61K 31/555; A61K 39/04; C12N 5/078; A61K 39/00

(52) U.S. Cl. CPC A61K 31/555; A61K 39/001; A61K 39/04; C12N 5/0634; A61K 39/0008; A61K 2039/54; A61K 2039/577; C12N 2501/71; A61K 2039/55511

(57) ABSTRACT

The invention is in the field of immunotherapy. More particularly, the invention provides a composition comprising a Heme Oxygenase-1 (HO-1) and antigens. Also provided herein are methods of administering the compositions of the invention by subcutaneous, intradermal or topical administration in a patient for inducing antigen-specific tolerance.

### FIELD OF THE INVENTION

The invention is in the field of immunotherapy.

More particularly, the invention provides a composition comprising a heme oxygenase-1 (HO-1) inducer and specific antigens. Also provided herein are methods of administering the composition of the invention by subcutaneous, intradermal or topical administration in a patient for inducing antigen-specific tolerance.

### BACKGROUND OF THE INVENTION

Autoimmune diseases, organ transplantation, allergy/asthma, immunogenic therapeutic proteins and gene therapy are immune-mediated pathophysiological situations in which common immune responses destroy tissues or eliminate a given molecule. Immune responses in said pathophysiological situations involve an overall increase in immunogenic dendritic cells (DCs), T effector/memory cells of the CD4⁺ and CD8⁺ lineages and antibody production along with decrease regulatory/suppressive mechanisms. Thus, they also share therapeutic approaches, such as historically immunosuppressors and more recently biological modifiers. Conventional clinical strategies for immunosuppression in disorders associated with an undesired immune response (e.g., autoimmune disease, graft rejection) are based on the long-term administration of broad acting immunosuppressive drugs, such as for example, cyclosporin A (CsA), FK506 (tacrolimus) or corticosteroids. Long-term use of high doses of these drugs can also have toxic side-effects. Moreover, even in those patients that are able to tolerate these drugs, the requirement for life-long immunosuppressive drug therapy carries a significant risk of severe side effects, including tumors, serious infections, nephrotoxicity and metabolic disorders. Accordingly, tolerance towards the antigens involved in these pathologies would greatly improve their treatment.

For instance, Type 1 diabetes (T1D) results from autoimmune destruction of insulin-secreting β cells. Observations in humans and in non-obese diabetic (NOD) mice (a valuable experimental model of T1D) have demonstrated the essential pathogenic role of CD8⁺ T cells during disease development, even shortly after diagnosis (reviewed by Tsai et al. (1)). Additionally, a robust correlation between recent-onset T1D and the presence of autoreactive β-cell-specific CD8⁺ T cells has recently been reported by our group (2) and by others (3-6).

One serious challenge in T1D treatment is that, at diagnosis, 70-90% of pancreatic β-cells have already been lost following antigen-specific T cells aggression, leaving a narrow therapeutic window (7). Systemic immunosuppression represents an important step towards curative T1D treatment, as it most likely improves disease by allowing proliferation of the remaining β cells or transdifferentiation of non-β cells. However, current immunosuppression carry the inherent problem of a lack of specificity, which results in undesirable side effects, such as increased risk of opportunistic infections (8). Thus, there is an urgent need for therapeutic strategies for selective targeting of autoreactive T cells. Efforts to tolerize antigen-specific naive T cells have been somewhat successful, but tolerance in clinically relevant activated antigen-specific CD8⁺ T cells has been obtained only with administration of massive doses of synthetic peptides emulsified in adjuvant (9-11), which are incompatible with clinical use.

Another immune-mediated pathophysiological situations is Multiple Sclerosis (MS), an autoimmune disease that causes demyelination of neurons and progressive damage to the central nervous system (CNS). The demyelination is thought to be mediated by auto-reactive T cells and B cells that cross the blood-brain barrier (BBB) and elicit an inflammatory process. This inflammation causes multiple discrete demyelinated plaques, which are primarily located in the white matter. Slowly progressive axonal injury advancing to axonal transection within the plaques contributes to the irreversible damage eventually seen in patients with advanced MS (Tabansky et al., 2015).

Accordingly, there is still a need for compositions capable of inducing antigen-specific tolerance in a patient in need thereof.

Heme Oxygenase-1 (HO-1) catalyzes the degradation of free heme in carbon monoxide (CO), biliverdin and iron and all of these molecules have anti-inflammatory and tolerogenic activities. Pharmacological inducers of HO-1 (such as Normosang®) are clinically approved for treatment of acute porphyrias in humans (12). HO-1 induction or genetic overexpression has potent anti-inflammatory effects in rodent models (as reviewed by Blancou et al. (13) and confers protection against autoimmune diseases (14-16). The precise mechanisms by which HO-1 acts have not yet been fully elucidated, but several studies have suggested that these mechanisms are at least partially dependent on antigen-presenting cells (APCs) (17, 18). Nevertheless, these results were based on long systemic treatments with HO-1 inducers without co-administration of a particular antigen of interest. This kind of treatment has the risk of attenuating all immune responses, not only those specific to said antigen.

### SUMMARY OF THE INVENTION

In a first aspect, the invention relates to a composition comprising (i) a Heme Oxygenase-1 (HO-1) inducer and (ii) at least one pathogenic antigen.

In a second aspect, the invention relates a composition of the invention for use in a method for inducing immune tolerance in a patient in need thereof.

In a third aspect, the invention relates to a composition of the invention for use in a method for inducing antigen-specific tolerance in a patient in need thereof.

In a fourth aspect, the invention relates to a composition of the invention for use in a method for preventing or reducing transplant rejection in a patient in need thereof.

In a fifth aspect, the invention relates to a composition of the invention for use in a method for preventing or treating autoimmune diseases, unwanted immune responses against proteins expressed in the course of gene therapy or therapeutic proteins, and allergies in a patient thereof.

In a sixth aspect, the invention relates to an in vitro or ex vivo method for generating a population of antigen-specific tolerogenic APCs, comprising a step of culturing a population of APCs with a culture medium comprising a heme oxygenase-1 (HO-1) inducer and said antigen of interest.

In a seventh aspect, the invention relates to a population of antigen-specific tolerogenic APCs.

### DETAILED DESCRIPTION OF THE INVENTION

The invention relates to the discovery that administration by intradermal injection of a composition comprising Heme Oxygenase-1 (HO-1) inducer and an antigen is effective to induce antigen-specific tolerance in a patient in need thereof. Following this treatment, a tolerogenic Antigen-Presenting Cell (APC) population overexpressing HO-1 (HO-1⁺ APCs) such as a monocyte-derived DC (MoDC) population overexpressing HO-1 is recruited in the draining lymph node in mice and non-human primates. These HO-1+ APCs inhibit the effector functions of pathogenic CD4⁺ and CD8⁺ T cells and the production of antibodies by B cells. Same results have been obtained in vitro with human HO-1⁺ APCs.

### Compositions of the Invention

Accordingly, a first aspect of the invention relates to a composition comprising or consisting of (i) a Heme Oxygenase-1 (HO-1) inducer and (ii) at least one pathogenic antigen.

As used herein, the term “Heme Oxygenase-1 (HO-1)” refers to an inducible rate-limiting enzyme which catalyzes group heme into carbon monoxide, iron and bilirubin. More particularly, HO-1 cleaves the α-meso carbon bridge of Heme B molecules by oxidation to yield equimolar quantities of biliverdin IXa, carbon monoxide (CO), and free iron. As used herein, the term “Heme” refers to a chemical compound of a type known as a prosthetic group consisting of a Fe2+ (ferrous) ion contained in the centre of a large heterocyclic organic ring called a porphyrin, made up of four pyrrolic groups joined together by methine bridges.

As used herein, the term “inducer” refers to substance capable of increasing the production of a protein, e.g., HO-1, in the body of a patient, using the patient's own endogenous (e.g., non-recombinant) gene that encodes the protein.

As used herein, the term “HO-1 inducer” refers to a substance capable of inducing HO-1 in a patient, e.g., any of the agents described herein, e.g., hemin, hematin, iron protoporphyrin, and/or cobalt protoporphyrin but also encompasses the HO-1 derivatives such as the heme degradation products (e.g. bilirubin, biliverdin, ferritin, desferoxamine, salicylaldehyde isonicotinoyl hydrazone, iron dextran and apoferritin).

In a particular embodiment, the present invention relates to a composition comprising or consisting of (i) a Heme Oxygenase-1 (HO-1) inducer and (ii) at least one pathogenic antigen wherein the HO-1 inducer does not consist of rapamycin.

HO-1 can be induced in a patient by any method known in the art. For example, production of HO-1 can be induced by hemin, by hematin, by iron protoporphyrin, or by cobalt protoporphyrin. A variety of non-heme agents including heavy metals, cytokines, hormones, nitric oxide, CoCl2, endotoxin and heat shock are also strong inducers of HO-1 expression (Otterbein et al., Am. J. Physiol. Lung Cell Mol. Physiol. 279:L1029-L1037, 2000; Choi et al., Am. J. Respir. Cell Mol. Biol. 15:9-19, 1996; Maines, Annu. Rev. Pharmacol. Toxicol. 37:517-554, 1997; and Tenhunen et al., J. Lab. Clin. Med. 75:410-421, 1970). HO-1 is also highly induced by a variety of agents and conditions that create oxidative stress, including hydrogen peroxide, glutathione depletors, UV irradiation and hyperoxia (Choi et al., Am. J. Respir. Cell Mol. Biol. 15: 9-19, 1996; Maines, Annu. Rev. Pharmacol. Toxicol. 37:517-554, 1997; and Keyse et al., Proc. Natl. Acad. Sci. USA 86:99-103, 1989).

In one embodiment, the HO-1 inducer is Cobalt protoporphyrin (CoPP) or protoporphyrin IX containing a ferric iron ion (Heme B) with a chloride ligand (Hemin).

In one embodiment, the HO-1 inducer is Hematin (trade name Panhematin®) or heme arginate (trade name NormoSang®).

Alternatively, HO-1 can be provided to a patient in need thereof by administering exogenous HO-1 directly to said patient. For instance, exogenous HO-1 protein can be directly administered to a patient by any method known in the art. Exogenous HO-1 can be directly administered in addition to, or as an alternative to the induction of HO-1 in the patient as described herein. The HO-1 protein can be delivered to a patient, for example, in liposomes, and/or as a fusion protein, e.g., as a TAT-fusion protein, and/or by gene therapy e.g. adenovirus vectors.

As used herein, the term “antigen” refers to a substance capable of binding to an antigen binding region of an immunoglobulin molecule (or antibody) or a T cell receptor (TCR). Thus, the term “antigen” includes, but is not limited to, antigenic determinants, haptens, and immunogens which may be proteins, polypeptides, peptides, small molecules (including oligopeptide mimics (i.e. organic compounds that mimic the antibody binding properties of the antigen)), carbohydrates e.g. polysaccharides, lipids, nucleic acids or combinations thereof. It should be further noted that an antigen according to the invention may be a protein which can be obtained by recombinant DNA technology or by purification from different tissue or cell sources. Such proteins are not limited to natural ones, but also include modified proteins or chimeric constructs, obtained for example by changing selected amino acid sequences or by fusing portions of different proteins. Alternatively, said antigen may be a synthetic peptide, obtained by Fmoc biochemical procedures, large-scale multipin peptide synthesis, recombinant DNA technology or other suitable procedures.

Within the context of the invention, compositions of the invention are useful in the prevention or treatment of unwanted immune responses, such as those involved in autoimmune diseases, immune reactions to therapeutic proteins, graft rejection and allergies.

Accordingly, antigens useful within the context of the invention are antigens associated with and/or involved in the disease/condition to be prevented or treated (also referred as “pathogenic antigen”). Therefore, the antigen of interest is selected from the group consisting of auto-antigens (self-antigens), allo-antigens, therapeutic proteins and allergens.

For instance, when the autoimmune disease is multiple sclerosis, the autoantigen is selected from the group consisting of myelin-related antigens (e.g. myelin basic protein (MBP) (e.g. MBP83-102 peptide), myelin oligodendrocyte glycoprotein (MOG) (e.g. MOG35-55 peptide) and proteolipid protein (PLP) (e.g. PLP139-151 peptide).

When the autoimmune disease is Type I diabetes (T1D), the autoantigen is selected from the group consisting of insulin, insulin precursor proinsulin (ProIns), glutamic acid decarboxylase 65 (GAD65), glial fibrillary acidic protein (GFAP), islet-specific glucose-6-phosphatase catalytic subunit-related protein (IGRP), insulinoma-associated antigen-2 (IA-2) and zinc transporter 8 (ZnT8).

In a particular embodiment, the present invention relates to a composition comprising or consisting of (i) a Heme Oxygenase-1 (HO-1) inducer and (ii) at least one pathogenic antigen wherein the pathogenic antigen does not consist of insulin.

When the autoimmune disease is rheumatoid arthritis, the autoantigen is type II collagen (CTII).

Other examples allergies where the body is overreacting to exogenous antigens called allergens. Among those allergens some are found in the food (e.g. crustacean, seeds, fruits, vegetables, milk, eggs, fish protein), some are non-food related (e.g. pollen, latex, cement, chrome) and some are against drugs (e.g. penicillin, aspirin, curare, morphine, vancomycine),

It is also intended that alloantigens include, but are not limited to, antigens expressed by the allograft, proteins expressed in the course of gene therapy (and also viral antigens issued from the viral vector used) as well as therapeutic proteins.

As such an “allograft” is a transplant between two individuals of the same species having two genetically different MHC haplotypes.

The term “therapeutic proteins” refers to proteins or peptides and their administration in the therapy of any given condition or illness. Therapeutic proteins relate to any protein or peptide, such as therapeutic antibodies, cytokines, enzymes or any other protein, that is administered to a patient. Examples of protein therapy relate to treatment of hemophilia via administration of plasma-derived or recombinant clotting factor concentrates (e.g. factor VIII or factor IX), the treatment of cancer or cardiovascular disease using monoclonal antibodies or the treatment of metabolic or lysosomal disease by enzyme replacement therapy.

In one embodiment of the invention, the composition is formulated for subcutaneous, intradermal or topical administration.

As used herein, the term “intradermal administration” refers to the delivery of the compositions to the regions of the dermis of the skin, although it will not necessarily be located exclusively in the dermis, which is the layer in the skin located between about 1.0 and about 2.5 mm from the surface in human skin. There may be a certain amount of variation between individuals and in different parts of the body. Generally, the dermis is reached by going approximately 1.5 mm below the surface of the skin, between the stratum corneum and the epidermis at the surface and the subcutaneous layer below, respectively. After administration, the compositions may be located exclusively in the dermis or it may also be present in the epidermis, hypodermis or in the draining lymph node.

Intradermal administration is a way of administering composition circumventing the use of long needles and the composition can be administered with devices that are reliable and easy to use. Moreover, skin is an excellent immune organ, because there is a high density of Langerhans cells, which are specialized dendritic cells. It is generally taken that intradermal administration of composition provides a more efficient uptake of antigen.

Any suitable device may be used for intradermal delivery, for example short needle devices such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496. U.S. Pat. No. 5,417,662. Compositions may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381 , U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911 , U.S. Pat. No. 5,383,851 , U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556 U.S. Pat. No. 4,790,824, U.S. Pat. No. 4,941,880, U.S. Pat. No. 4,940,460, WO 97/37705 and WO 97/13537. Also suitable are ballistic powder/particle delivery devices which use compressed gas to accelerate composition in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Preferably, a needle-free jet injector service is used, such as that published in WO 01/05453, WO 01/05452, WO 01/05451 , WO 01/32243, WO 01/41840, WO 01/41839, WO 01/47585, WO 01/56637, WO 01/58512, WO 01/64269, WO 01/78810, WO 01/91835, WO 01/97884, WO 02/09796, WO 02/34317. More preferably said device is pre-filled with the liquid composition formulation.

Another suitable administration route is the topical administration (also called epicutaneous route).

As used herein, the terms “topical administration” and “epicutaneous route” refer to the delivery of the compositions by application of this composition on the skin (or to a mucous membrane, also called mucosa, lining all body passages that communicate with the exterior such as the respiratory, genitourinary, and alimentary tracts, and having cells and associated glands that secrete mucous). The topical administration does not require the use of a needle, syringe or of any other means to perforate or to alter the integrity of the superficial layer of the epidermis. The active substance is maintained in contact with the skin for period of time and under conditions sufficient to allow the active substance to penetrate into the stratum corneum of the epidermis.

Any suitable device may be used for example, skin patch device, gel or ointment.

As used herein, the term “skin patch device” (also called “dermal patch”) refers to is a medicated adhesive patch that is placed on the skin to deliver a medication into the skin.

As used herein, the term “gel” refers to a colloid in a more solid form than a solution. A gel is also a jelly-like material formed by the coagulation of a colloidal liquid. Many gels have a fibrous matrix and fluid filled interstices. Gels are viscoelastic rather than simply viscous and can resist some mechanical stress without deformation.

As used herein, the term “ointment” means a semisolid, oil-based topical formulation. Examples of ointments include essentially non-aqueous mixtures of petrolatum, lanolin, polyethylene glycol, plant or animal oils, either hydrogenated or otherwise chemically modified. An ointment may also contain a solvent in which an active agent is either fully or partially dissolved.

The composition may be formulated for repeated subcutaneous, intradermal or topical administration, for example at alternating successive sites. The composition may be administered, for example by subcutaneous, intradermal or topical injection at an administration site, in successive doses given at a dosage interval, for example of between one hour and one month, over a dosage duration, for example of at least 2 weeks, 2 months, 6 months, 1, 2, 3, 4, or 5 years or longer.

The invention also relates to combination of the compositions according to the invention and the delivery device with which it is being delivered subcutaneously, intradermally or topically. Hence, the invention also relates to a kit comprising a composition according to the invention and a delivery device suitable for subcutaneous, intradermal or topical delivery of said composition. Even more preferred are kits in which the composition is already present inside the delivery device, which enables a health worker to easily administer the composition to the patient.

As with all compositions of the invention, the immunologically effective amounts of the antigens must be determined empirically. Factors to be considered include the tolerance, whether or not the antigens will be complexed with or covalently attached to a carrier protein or other carrier and the number of tolerizing dosages to be administered. Such factors are known in the art and it is well within the skill of immunologists to make such determinations without undue experimentation.

The antigen and the HO-1 inducer can be present in varying concentrations in the composition of the invention. Typically, the minimum concentration of said substances is an amount necessary to achieve its intended use, while the maximum concentration is the maximum amount that will remain in solution or homogeneously suspended within the initial mixture. For instance, the minimum amount of a therapeutic agent is one which will provide a single therapeutically effective dosage. For bioactive substances, the minimum concentration is an amount necessary for bioactivity upon reconstitution and the maximum concentration is at the point at which a homogeneous suspension cannot be maintained. In the case of single-dosed units, the amount is that of a single therapeutic application. Generally, it is expected that each dose will comprise 1-100 μg/kg of antigen, for example 25 or 50 μg/kg. Moreover, it is expected that each dose will comprise 1-8 mg/kg of HO-I inducer, for example 3 or 4 mg/kg (i.e. a dose inferior to the dose of 12 mg/kg administered systemically). The preferred amount of the substances varies from substance to substance but is easily determinable by one of skill in the art.

Another aspect of the invention relates to a composition suitable for subcutaneous, intradermal or topical administration in a patient suffering from or at risk of a condition comprising (i) a HO-1 inducer and (ii) at least one antigen involved in said condition.

Another aspect of the invention relates to composition suitable for subcutaneous, intradermal or topical administration comprising (i) a HO-1 inducer and (ii) at least one pathogenic antigen.

Another aspect of the invention relates to a composition for administration to the subcutaneous or intradermal compartment of a patient's skin suffering from or at risk of a condition comprising (i) a HO-1 inducer and (ii) at least one pathogenic antigen involved in said condition, so that the composition induces antigen-specific tolerance in said patient when delivered to the subcutaneous or intradermal compartment.

Another aspect of the invention relates to a composition suitable for induction of antigen-specific tolerance in a patient suffering from or at risk of a condition comprising (i) a HO-1 inducer and (ii) at least one antigen involved in said condition.

In one embodiment, the present invention relates to a composition comprising or consisting of (i) a Heme Oxygenase-1 (HO-1) inducer and (ii) at least one pathogenic antigen involved in a condition for use in a method for inducing antigen-specific tolerance in a patient in need thereof, wherein the composition is administrated topically or intradermally to a patient's skin suffering from or at risk of said condition.

In a further aspect, the composition of the invention can be contained in a vector, such as a membrane or lipid vesicle (e.g. a liposome), or contained in a nanocarrier such as synthetic nanocarriers, lipid nanoparticles, metallic nanoparticles such as gold nanoparticles, polymeric nanoparticles (such as polymeric nanoparticles comprising polymer that is a non-methoxy-terminated, pluronic polymer, polyester (such as poly(lactic acid), poly(glycolic acid), poly(lactic-co-glycolic acid) or polycaprolactone), polyester coupled to a polyether (such as polyethylene glycol or polypropylene glycol), polyamino acid, polycarbonate, polyacetal, polyketal, polysaccharide, polyethyloxazoline or polyethyleneimine), surfactant-based emulsions, dendrimers, buckyballs, nanowires, virus-like particles or peptide or protein particles such as described WO 2012/149265.

### Methods for Obtaining a Population of Antigen-Specific Tolerogenic APCs

In another aspect, the invention relates to an in vitro or ex vivo method for generating a population of antigen-specific tolerogenic APCs, comprising a step of culturing a population of APCs with a culture medium comprising a heme oxygenase-1 (HO-1) inducer and said antigen of interest.

As used herein, the terms “antigen-presenting cell” (APC) refer to a class of immune cells capable of internalizing and processing an antigen, so that antigenic determinants are presented on the surface of the cell as MHC-associated complexes, in a manner capable of being recognized by the immune system (e. g., MHC class I restricted cytotoxic T lymphocytes and/or MHC class II restricted helper T lymphocytes). The two requisite properties that allow a cell to function as an APC are the ability to process endocytosed antigens and the expression of MHC gene products. Examples of APC include dendritic cells (DC), mononuclear phagocytes (e. g. macrophages), B lymphocytes, Langerhans cells of the skin and, in humans, endothelial cells.

As used herein, the term “culture medium” refers to any medium capable of supporting the growth and the differentiation of APCs into tolerogenic APCs. Typically, it consists of a base medium containing nutrients (a source of carbon, amino acids), a pH buffer and salts, which can be supplemented with growth factors and/or antibiotics. Typically, the base medium can be RPMI 1640, DMEM, IMDM, X-VIVO or AIM-V medium, all of which are commercially available standard media.

Preferred media formulations that will support the growth and the differentiation of APCs into tolerogenic APCs include chemically defined medium (CDM). As used herein, the term “chemically defined medium” (CDM) refers to a nutritive solution for culturing cells which contains only specified components, preferably components of known chemical structure. A chemically defined medium is a serum-free and feeder-free medium.

The step of culturing a population of APCs with a culture medium comprising a HO-1 inducer and at least one antigen of interest shall be carried out for the necessary time required for the internalization of the HO-1 inducer and the antigen in the APCs and the presentation of said antigen by said APCs. Typically, the culture of a population of APCs with the culture medium shall be carried from 3, 6, 12 hours to 1 day or more.

The antigen of interest is selected from the group consisting of auto-antigens (self-antigens), allo-antigens, therapeutic proteins and allergens as previously described.

The HO-1-inducer is a substance capable of inducing HO-1 in a patient as previously described.

In one embodiment, the HO-1 inducer is Cobalt protoporphyrin (CoPP) or protoporphyrin IX containing a ferric iron ion (Heme B) with a chloride ligand (Hemin).

In one embodiment, the HO-1 inducer is Hematin (trade name Panhematin®) or heme arginate (trade name NorrnoSang®).

Alternatively, HO-1 can be provided to the APCs by administering exogenous HO-1. The means by which the vector carrying the gene may be introduced into the cells include, but are not limited to, microinjection, electroporation, transduction, or transfection using DEAE-dextran, lipofection, calcium phosphate or other procedures known to one skilled in the art.

In one embodiment, the APC is a dendritic cell.

In one particular embodiment, the antigen-presenting cells (APC) are human dendritic cells or monocytes (particularly those obtained from the patient to be treated).

Populations of tolerogenic APCs specific for an antigen associated with the disease to be treated (pathogenic antigen) should be obtained. Therefore, the antigen of interest is selected from the group consisting of auto-antigens, allo-antigens and allergens.

In case dendritic cells (DCs) are used, the APCs can be prepared as follows. Lymphocytes are isolated from peripheral blood by Ficoll method; adherent cells are separated from non-adherent cells; the adherent cells are then cultured in the presence of GM-CSF and IL-4 to induce DCs; and said DCs are cultured with a HO-1 inducer and the antigen of interest to obtain antigen-specific tolerogenic DCs. The resulting DCs can then be re-administrated to the patient to be treated. Such methods are described in WO93/208185 and EP0563485, which are incorporated by reference.

Another aspect of the invention relates to a population of antigen-specific tolerogenic APCs.

In one embodiment, the antigen-specific tolerogenic APCs are obtained by the method of the invention described above.

In one embodiment, the antigen-specific tolerogenic APCs are MHC-II⁺ CD14⁺ CD11c⁺ cells.

The invention also relates to a pharmaceutical composition comprising a population of antigen-specific tolerogenic APCs as well as to a population of antigen-specific tolerogenic APCs or a pharmaceutical composition comprising thereof for use as drug.

### Methods for Obtaining a Population of Antigen-Specific Regulatory T Cells by Using a Population of Antigen-Specific Tolerogenic APCs

Another aspect of the invention relates to a method for obtaining a population of regulatory T cells specific for an antigen comprising a step of culturing a population of regulatory T cells with a population of tolerogenic APCs specific for said antigen.

The invention also relates to a method for obtaining a population of regulatory T cells specific for an antigen, comprising the steps of:
culturing a population of APCs with a culture medium comprising a heme oxygenase-1 (HO-1) inducer and said antigen in order to obtain a population tolerogenic APCs specific for said antigen,
optionally, isolated said population of tolerogenic APCs specific for said antigen, and
culturing a population of regulatory T cells with said population of antigen-specific tolerogenic APCs.

In one embodiment, the population of regulatory T cells is a population of CD4+CD25+ regulatory cells.

In a particular embodiment, the population of regulatory T cells is a population of naïve CD4+CD25+CD45RA+ regulatory T cells.

In one embodiment, the population of regulatory T cells is a population of CD8+CD45RC^{low or-} regulatory T cells.

Within the context of the application, the term “a population of regulatory T cells” refers to a population of T cells characterized by an ability to suppress or downregulate immune reactions mediated by effector T cells, such as effector CD4+ or CD8+ T cells.

In one embodiment, the regulatory T cells are human regulatory T cells (particularly those obtained from the patient to be treated).

The population of regulatory T cells that serve as starting material may be isolated according to any technique known in the art. For instance, the population of regulatory T cells may be obtained from various biological samples containing lymphocytes. Typically, they are isolated from peripheral blood. They may be isolated by a combination of negative and positive selection with beads labelled with different ligands (eg, CD4 and CD25). Such labelled cells may then be separated by various techniques such as cell sorting. The resulting regulatory T cells can then be re-administrated to the patient to be treated.

Another aspect of the invention relates to a population of antigen-specific regulatory T cells.

In one embodiment, the antigen-specific regulatory T cells are obtained by the method of the invention described above.

The invention also relates to a pharmaceutical composition comprising a population of antigen-specific regulatory T cells as well as to a population of antigen-specific regulatory T cells or a pharmaceutical composition comprising thereof for use as drug.

### Therapeutic Methods and Uses

The invention provides methods and compositions (such as compositions or the population of cells of the invention) for use in a method for inducing immune tolerance, and more particularly, antigen-specific tolerance, in a patient in need thereof.

The invention also provides methods and compositions for use in a method for preventing or reducing transplant rejection in a patient in need thereof.

The invention further provides methods and compositions for use in a method for preventing or treating autoimmune diseases, unwanted immune response against therapeutic proteins and allergies in a patient in need thereof.

In a first aspect, the invention relates to a composition of the invention for use in a method for inducing immune tolerance in a patient in need thereof.

As used herein, the terms “immune tolerance” or “tolerogenic immune response” refers to the absence of pathogenic process induced by the immune system to antigens that have the capacity to elicit an immune response. Tolerogenic immune responses include any reduction, delay or inhibition in CD4+ T cell, CD8+ T cell or B cell proliferation and/or activity (anergy) and/or migration to targeted tissues. Tolerogenic immune responses can also include any response that leads to the stimulation, induction, production or recruitment of regulatory cells, such as tolerogenic dendritic cells, CD4+ Treg cells, CD8+ Treg cells, Breg cells, etc. In some embodiments, the tolerogenic immune response is one that results in the conversion to a regulatory phenotype characterized by the production, induction, stimulation or recruitment of cells. Tolerogenic immune responses may also include a reduction in antibody production.

As used herein, the term “immune response” includes T cell mediated and/or B cell mediated immune responses. Exemplary immune responses include T cell responses, e.g., cytokine production and cellular cytotoxicity, in addition, the term immune response includes immune responses that are indirectly effected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Th1 and Th2 cells); antigen presenting cells (e.g. professional antigen presenting cells such as dendritic cells (DCs)); natural killer cells; myeloid cells, such as macrophages, eosinophils, mast cells, basophils, and granulocytes.

As used herein, the term “antigen-specific immune tolerance” refers to a specific immune tolerance to a given antigen. It is an active antigen-dependent process in response to the antigen. Like immune response, tolerance is specific and like immunological memory, it can exist in T-cells, B cells or both.

In one embodiment, the composition of the invention is capable of inhibiting or reducing the pathogenic process induced by the immune responses, and in particular is capable of inducing a tolerization of antigen-specific T cells such as antigen-specific CD8⁺ and CD4⁺ memory T-cells as well as activated antigen-specific cytotoxic T cells (CTLs) and antigen-specific B cells producers of antibodies directed against a given antigen, compared to that obtained with the antigen administered without HO-1 inducer and/or the HO-1 inducer administered locally without the antigen. Specifically said antigen-specific T cells such as CD8⁺ and CD4⁺ memory T-cell tolerization or activated CTLs is further capable of inducing immune tolerance against said antigen without affecting immune responses directed against other antigens. Specifically said antigen-specific T-cell tolerization involves the induction of HO-1⁺ tolerogenic APCs such as MoDCs in draining lymph node (DLN) cells (MHC-II⁺CD11b⁺Ly6⁺F4/80⁺CD11c^{low}CD64+FcεR1⁺ cells in mice and MHC-II⁺ CD14⁺ CD11c⁺ in baboons and humans).

By “patient in need thereof” is meant an individual suffering from or susceptible of suffering from transplant rejection, an autoimmune disease, alloimmune response or allergy to be treated. The individuals to be treated are mammals, preferably human beings.

In a second aspect, the invention relates to a composition of the invention for use in a method for preventing or reducing transplant rejection in a patient in need thereof.

As used herein, the term “preventing or reducing transplant rejection” is meant to encompass prevention or inhibition of immune transplant rejection, as well as delaying the onset or the progression of immune transplant rejection. The term is also meant to encompass prolonging survival of a transplant in a patient, or reversing failure of a transplant in a patient. Further, the term is meant to encompass ameliorating a symptom of an immune transplant rejection, including, for example, ameliorating an immunological complication associated with immune rejection, such as for example, interstitial fibrosis, chronic graft arteriosclerosis, or vasculitis.

As used herein, the term “transplant rejection” encompasses both acute and chronic transplant rejection. “Acute rejection” is the rejection by the immune system of a tissue transplant recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporin, anti-CD40L monoclonal antibody and the like. “Chronic rejection” generally occurs in humans within several months to years after engraftment, even in the presence of successful immunosuppression of acute rejection. Fibrosis is a common factor in chronic rejection of all types of organ transplants.

The term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species, for example, a baboon heart being transplanted into a human host.

In one embodiment the donor of the transplant is a human. The donor of the transplant can be a living donor or a deceased donor, namely a cadaveric donor.

In one embodiment, the transplant is an organ, a tissue or cells.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus. As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

In a particular embodiment of the invention, the transplant is a cardiac allotransplant.

As used herein, the term “cells” refers to a composition enriched for cells of interest, preferably a composition comprising at least 30%, preferably at least 50%, even more preferably at least 65% of said cells.

In certain embodiments the cells are selected from the group consisting of multipotent hematopoietic stem cells derived from bone marrow, peripheral blood, or umbilical cord blood; or pluripotent (i.e. embryonic stem cells (ES) or induced pluripotent stem cells (iPS)) or multipotent stem cell-derived differentiated cells of different cell lineages such as cardiomyocytes, beta-pancreatic cells, hepatocytes, neurons, etc . . .

In one embodiment, the cell composition is used for allogeneic hematopoietic stem cell transplantation (HSCT) and thus comprises multipotent hematopoietic stem cells, usually derived from bone marrow, peripheral blood, or umbilical cord blood.

HSCT can be curative for patients with leukemia and lymphomas. However, an important limitation of allogeneic HCT is the development of graft versus host disease (GVHD), which occurs in a severe form in about 30-50% of humans who receive this therapy.

Compositions of the invention are useful in a method for preventing or reducing Graft-versus-Host-Disease (GvHD).

Accordingly, in one embodiment, the patient in need thereof is affected with a disease selected from the group consisting of acute myeloid leukemia (AML); acute lymphoid leukemia (ALL); chronic myeloid leukemia (CML); myelodysplasia syndrome (MDS)/myeloproliferative syndrome; lymphomas such as Hodgkin and non-Hodgkin lymphomas, chronic lymphatic leukemia (CLL) and multiple myeloma.

In a third aspect, the invention relates to a composition of the invention for use in a method for preventing or treating autoimmune diseases, unwanted immune responses against proteins expressed in the course of gene therapy or therapeutic proteins and allergies in a patient thereof.

As used herein, the terms “prevent”, “preventing” and “prevention” refer to the administration of therapy to an individual who may ultimately manifest at least one symptom of a disease, disorder, or condition, but who has not yet done so, to reduce the chance that the individual will develop the symptom of the disease, disorder, or condition over a given period of time. Such a reduction may be reflected, for example, in a delayed onset of the at least one symptom of the disease, disorder, or condition in the patient.

As used herein, the terms “treat”, “treating” or “treatment” refers to the administration of therapy to an individual in an attempt to reduce the frequency and/or severity of symptoms of a disease, defect, disorder, or adverse condition of a patient.

As used herein, the term “autoimmune disease” refers to a disease in which the immune system produces an immune response (for example, a B-cell or a T-cell response) against an antigen that is part of the normal host (that is an auto-antigen), with consequent injury to tissues. In an autoimmune disease, the immune system of the host fails to recognize a particular antigen as “self” and an immune reaction is mounted against the host's tissues expressing the antigen.

Exemplary autoimmune diseases affecting humans include rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (for example, Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenic purpura and the like.

As used herein, the term “unwanted immune response against a therapeutic protein” refers to any unwanted immune reaction directed to proteins expressed in the course of gene therapy, and/or therapeutic proteins, such as factor VIII (hemophilia A) and other coagulation factors, enzyme replacement therapies, monoclonal antibodies (e.g. natalizumab, rituximab, infliximab), polyclonal antibodies (ATG), hormones (insulin) or cytokines (e.g. IFN).

For instance, this approach can indeed be applied to suppress an immune response, especially to prevent immune reactions to specific proteins when their expression is restored by gene therapy in individuals with corresponding genetic deficiencies. Thus, an immunogenic composition according to the invention may be used to prevent immune reactivity towards proteins normally absent in the patient due to mutations, while their reconstitution is achieved by gene therapy.

Moreover, protein therapy is an area of medical innovation that is becoming more widespread, and involves the application of proteins, such as enzymes, antibodies or cytokines, directly to patients as therapeutic products. One of the major hurdles in delivery of such medicaments involves the immune responses directed against the therapeutic protein themselves. Administration of protein-based therapeutics is often accompanied by administration of immune suppressants, which are used in order to facilitate a longer lifetime of the protein and therefore increased uptake of the protein into the cells and tissues of the organism. General immune suppressants can however be disadvantageous due to the unspecific nature of the immune suppression that is carried out, resulting in unwanted side effects in the patient. Therefore, this approach can be applied to suppress an immune response against therapeutic proteins and peptides, such as therapeutic antibodies, cytokines, enzymes or any other protein administered to a patient.

As used herein, the term “allergy” or “allergies” refers to a disorder (or improper reaction) of the immune system. Allergic reactions occur to normally harmless environmental substances known as allergens; these reactions are acquired, predictable, and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

Another aspect of the invention relates to a method for inducing immune tolerance in a patient in need thereof, comprising a step of administering subcutaneously, intradermally or topically to said patient an effective amount of a composition of the invention as described above.

Another aspect of the invention relates to a method for inducing antigen-specific tolerance in a patient in need thereof, comprising a step of administering subcutaneously, intradermally or topically to said patient an effective amount of a composition of the invention as described above.

Another aspect of the invention relates to a method for preventing or reducing transplant rejection in a patient in need thereof, comprising a step of administering subcutaneously, intradermally or topically to said patient an effective amount of a composition of the invention as described above.

Another aspect of the invention relates to a method for preventing or treating autoimmune diseases, unwanted immune responses against proteins expressed in the course of gene therapy or therapeutic proteins, and allergies in a patient thereof comprising a step of administering subcutaneously, intradermally or topically to said patient an effective amount of a composition of the invention as described above.

In one embodiment, the present invention relates to a method for inducing antigen-specific tolerance in a patient in need thereof, comprising a step of administering topically or intradermally to a patient's skin suffering from or at risk of a condition a composition comprising or consisting of (i) a Heme Oxygenase-1 (HO-1) inducer and (ii) at least one pathogenic antigen involved in said condition.

The invention will be further illustrated by the following figures and examples. However, these examples and figures should not be interpreted in any way as limiting the scope of the present invention.

### FIGURES

FIG. 1A-D: Intradermal injection of CoPP and autoantigen induces HO-1 in MHC-II⁺ cells in the draining LN, and tolerizes CTLs. (A, B) Twenty-four hours after intradermal injection of ovalbumin alone or with HO-1 inducer (CoPP), the proportion of HO-1⁺ cells among MHC-II⁺ cells was determined in splenocytes and draining LNs by FACS. Data are shown as a representative density plot (A), and as mean frequencies±s.e.m. of three independent experiments (n>3 mice/group) (B). (C, D) The day after intradermal treatment with the indicated conditions, Rip-OVA^{high} mice were transferred with 0.5×10⁵ autoreactive CTLs. Mice were monitored for diabetes development. HSA: human serum albumin. Data are shown from the indicated numbers of mice pooled from at least two independent experiments (C). At 6-8 days after treatment, histological analysis was used to determine the extent of insulitis in two different experiments (n=5-6 mice/group), with >150 islets analyzed for both groups (D). *P<0.05; **P<0.01; ***P<0.001.

FIG. 2: Intradermal injection of CoPP and autoantigen induces HO-1⁺ blood-MoDCs. In C57/B6 mice, the cell populations in draining LN were analyzed 24 hours after intradermal injection of ovalbumin (OVA) alone or with HO-1 inducer (CoPP). The number of MHC-II⁺F4/80⁺CD11b⁺Ly6C⁺ cells in the draining LN (n=4 mice/group) is reported.

FIG. 3A-B: HO-1⁺ moDCs tolerize autoreactive CTLs. Rip-OVA^{high} mice were transferred with 0.5×10⁵ autoreactive CTLs, and were co-cultured for 4 hours with MHC-II⁺ or CD11b⁺CD11c^{low}Ly6C^{high}F4/80⁺ cells isolated from the draining LN of mice intradermally immunized with OVA or CoPP-OVA (A). Mice were monitored for diabetes development.

Data shown are from the indicated numbers of mice pooled from three independent experiments. (B) Twenty-four hours after intradermal treatment with the indicated conditions, F4/80-sorted cells from the draining LNs were stimulated with LPS for 24 h. Supernatants were analyzed for IL-12 and IL-10 contents by ELISA. Data show mean±s.e.m. of three experiments.

FIG. 4A-D: Intradermal injection of CoPP plus autoantigen reduces CTL velocity.

Rip-OVA^{high} mice were intradermally immunized with OVA or CoPP-OVA. Twenty-four hours after, mice were either transferred with 0.5×10⁵ autoreactive CD45.1⁺ CTLs (A-B) or MHC-II⁺ cells were isolated from draining LN for in vitro experiments (C, D). (A) On day 2 or 6, in vivo CTL assay was performed and specific lysis was determined 12 hours after target cell transfer; data show mean±s.e.m. of three experiments (n≧6 mice/group). (B) Two days after CD45.1⁺ CTLs transfer, absolute numbers of CD8⁺CD45.1⁺ cells were determined in spleen, draining LNs, and PLNs; data show mean±s.e.m. of three experiments (n=9 mice/group). For velocity measurement (C) and transwell migration assay (D), data show mean±s.e.m. of three independent experiments. ***P<0.001.

FIG. 5A-C: Phenotype of human monocyte following HO-1 inducer treatment. (A) Human PBMC were analyzed for HO-1 expression 4 hours after in vitro hemin treatment.

Data show mean frequencies±s.e.m. of five donors. Human monocytic THP-1 cells were analyzed for HO-1 expression 16 hours after in vitro CoPP treatment (B) or clinically relevant hemin treatment (C). Data shown as mean±s.e.m. of three independent experiments (A, B, C). *P<0.05.

FIG. 6A-G. Intradermal injection of CoPP and autoantigen protect against EAE by inhibiting pathogenic T-cells migration to the CNS. (a) Mice were induced for EAE and treated or not either starting at the time of EAE induction (“treatment before onset”) or starting after the appearance of the first clinical signs (“treatment after onset”) three times three days apart. Clinical scores were measured every day and are shown as mean±SEM. (b, c) Representative hematoxylin and eosin spinal cord staining are shown (b). The quantification of the infiltrate was calculated by manual measurement of areas after clipping. Using a matrix each infiltrating cell is detectable like a one black pixel (c). One representative experiment out of two is presented (n=4 mice/group). Treatment before and after onset is a pool of three and seven experiments respectively. (d,e) The impact of CoPP treatment on T cell proliferation (day 0 and 3) was evaluated in C57/B6 CD45.1/1 mice immunized with MOG₃₅₋₅₅ peptide that received 6×10⁶ CFSE-loaded 2D2 anti-MOG CD45.2/2 CD4⁺ cells. Data are presented as representative dot plots (d), and as the percentage of proliferating cell±s.e.m. (n=7-8 mice/group) (e). (f, g) CD4⁺ T-cells from CNS or DLN were harvested and stained with MOG₃₈₋₄₉-APC tetramer. Data are presented as representative dot plots (f), and as number of cells per organ±s.e.m. (n=7-8 mice/group) (g).

FIG. 7A-F. Treatment with clinical hemin in non-human primates induces HO-1 in MHC-II⁺ cells in the draining LN and extinguish DTH reaction. (a-d) HO-1 induction with Normosang® was evaluated either in vivo after intradermal immunization in Baboons. Twenty-four hours after intradermal injection of clinical grade hemin (Normosang®), the proportion of HO-1⁺ (a) or CD14⁺CD11c⁺ (b) in MHC-II⁺ cells was determined by intracellular staining in the DLN and in the non-DLN (nDLN) of treated or non-treated baboons. Data show representative FACS profile (a,b) and frequencies (c) with one baboon/condition. (d) The expression of HO-1 was evaluated in MHC-II⁺CD11c⁺CD14⁺ or CD14⁻ cells. (e) Four baboons were immunized with BCG and challenged with tuberculin for DTH assessment before and after Normosang (hemin) treatment. (f) Erythema was measured every days following intradermal injection of tuberculin in all animals.

FIG. 8. Intravenous injections of CoPP and autoantigen do not protect against induced diabetes. The day after intravenous CoPP and autoantigen (“CoPP OVA IV”) or autoantigen alone (“OVA IV”), Rip-OVA^{high} mice were transferred with 0.5×10⁵ autoreactive CTLs. Mice were monitored for diabetes development. Results are pooled from two independent experiments. Curves are not statistically different.

### EXAMPLES

### Example 1: Tolerization of Ongoing CTL Response in Type 1 Diabetes (T1D) by Monocyte-Derived Dendritic Cells Induced by Intradermal Injection of Heme-Oxygenase-1 Inducer

Material & Methods

Cells:

Autoreactive CTL generation: Autoreactive CTLs were generated as described previously (34). Briefly, CD8⁺ cells were isolated by magnetic selection (Miltenyi Biotech) from OVA-specific class I-restricted T cells (OT-I) mice (35) spleen and lymph node single-cell suspensions. 1×10⁶ purified OT-I CD8⁺ T cells were stimulated with 5×10⁶ mitomycin-treated and ovalbumin (257-264) peptide-loaded syngeneic spleen cells in 2 ml complete DMEM high glucose with stable glutamine (PAA) supplemented with 10% FCS (Eurobio) containing, 5 ng/ml IL-2 (Roche Applied Science), and 20 ng/ml IL-12 (R&D Systems). On day 3, the cultures were split into four aliquots and fed with fresh medium containing IL-2. On day 6, cells were collected and washed with culture medium at three times.

Isolation of murine APCs and co-culture with autoreactive CTLs: For MHC-II⁺ and F4/80⁺ cells isolation, skin-draining LNs were removed 24 hours after intradermal injection in the back or in the ear. Single-cell suspensions were prepared by enzymatic lymph node disaggregation with collagenase D (Sigma-Aldricht). Cells were stained with anti-MHC-II-FITC (clone AF6-120.1, BD pharmingen) or anti-F4/80-PE (clone BM8, eBioscience) monoclonal antibodies as primary labeling reagent and respectively with anti-FITC or anti-PE microbeads (Miltenyi) as secondary reagent. After magnetic separation, we checked that purity was >82% for CD11b⁺CD11c^{low}Ly6C^{high}F4/80⁺ cells in CoPP-OVA immunized mice. These cells were co-cultured with autoreactive CTLs (respectively 1:1 and 1:5 APC to CTL ratio) overnight for velocity measurement and transwell migration assay.

Human THP-1 monocytic cell line and co-culture with CD8⁺ T cells clones: THP-I cells were cultured in RPMI 1640 (Gibco) supplemented with 10% fetal bovine serum (Eurobio), 1% glutamine (Gibco), 50 IU/mL penicillin 50 IU/mL streptomycin (Gibco) and 200 nM PMA (Sigma-Aldrich) under standard conditions as previously described (36).

For co-culture experiments with CD8⁺ T cells clones, THP-1 were treated with hemin (25 μM or 50 μM) or CoPP (12.5 μM or 25 μM) overnight, pulsed 30 min at 37° C. with 5 μM of MUCI (950-958) or NYESO-1 (156-165) in culture medium, thoroughly washed and cocultured respectively with HLA-A*0201/MUC1(950-958)-specific T-cell clone (N5.14) (37) or HLA-A*0201/NYESO-1 156-165)-specific CD8⁺ T-cell clone (M117.167) (38) at ratio 1:1 overnight for velocity measurement.

Human and non-human primate PBMC: Human PBMCs were obtained at the Etablissement Français du Sang in Nantes from blood of healthy donors. After Ficoll-Paque density gradient centrifugation (GE Healthcare), PBMC were cultured in RPMI 1640 (Gibco) supplemented with 10% fetal bovine serum (Eurobio), 1% glutamine (Gibco), 50 IU/mL penicillin 50 IU/mL streptomycin (Gibco) in presence or not of CoPP or hemin during 4 hours.

Animals:

Non-Human primates: Baboons (Papio anubis, from the CNRS Primatology Center, Rousset, France) were negative for all quarantine tests. Animals were housed at the large animal facility of our laboratory following the recommendations of the Institutional Ethical Guidelines of the Institut National de la Santé Et de la Recherche Médicale, France. All experiments were performed under general anaesthesia with Zoletil (Virbac, Carron, France). Three Baboons were injected intradermally in the inguinal fold with respectively 6.25 mg (500 μL), 12.5 mg (500 μL) or 25 mg (1 mL) of clinical hemin (Normosang®). A non-treated baboon has served as control. Inguinal lymph nodes were surgically removed 24 hours after intradermal injection. Single-cell suspensions for flow cytometry analysis were prepared by enzymatic lymph node disaggregation with Collagenase D (Sigma-Aldricht).

Generation of pIi-TTA-TetO-HO-1 transgenic NOD mice: pIi-TTA mice were a kind gift from Christophe Benoist (21). For generation of the TetO-HO-1 mice, the human HO-1 cDNA, the human β-globin intron located upstream of the cDNA sequence and the bovine growth hormone polyA located downstream of the cDNA was cloned at the Not-I/Xho-I sites into pBluKSM-tet-O-CMV vector containing the Tet-responsive-element (TRE) downstream the minimal CMV promoter followed by the human β-globin intron and the bovine growth hormone polyA. Transgenic mice were generated by pronuclear microinjection of CBA/C57BL6 eggs with the XhoI-NotI fragment of the vector described. Seventeen different founders were carrying the transgene as tested by PCR and southern blot. Of the 17 lines, 3 founders contained high copies of the hHO-1 cDNA. One of these was further analyzed by crossing with actin-rtTA mice. hHO-1 expression was confirmed by western blotting. Finally, both strains pIi-TTA and TetO-HO-1 were backcrossed to NOD/LtJ mice (Charles River, France) for at least twelve generations. Only females where used in experiments.

Other mice: The Rip-OVAhigh transgenic mice (39) express OVA in pancreatic islets and the OT-I CD45.1⁺ transgenic mice express a TCR-specific for the H2Kb restricted epitope of OVA and the CD45.1 congenic marker. Rip-OVAhigh and OT-I CD45.1⁺ and C57/BL6 mice were obtained respectively through Jackson Laboratory, Charles River and Janvier. All animal breeding and experiments were performed under conditions in accordance with the Inserm and European Union Guidelines.

Doxycycline treatment: Doxycycline hyclate powder (Sigma-Aldricht) was diluted in drinking water at different concentration (200 μg/mL up to 800 μg/mL) and protected from light. HO-1 transgenic mice have been treated during 3 days for HO-1 expression analysis and up to 200 days for diabetes incidence experiments.

Intradermal immunization of mice and diabetes induction: Eight to ten week-old Rip-OVAhigh mice received two intradermal injections in the back with 70 μg of CoPP (Livchem) prepared as described and 20 μg of endofree ovalbumin (Hyglos) in 10 μl. One hundred and forty micrograms of MnPP (Livchem) prepared as described (18) was added to the preparation for HO-1-inhibition experiments. Forty micrograms of Alexa Fluor® 488 ovalbumin (Molecular Probes) has been used for phagocytosis assay. For diabetes induction, mice were injected i.v. the following day with 0.5×10⁵ autoreactive cytotoxic OT-I CD8⁺ T cells (purity >95%) previously co-cultured or not with MHC-II⁺ or F4/80⁺ cells during 4 hours.

Diabetes follow-up: Diabetes monitoring has been done by urine glycosuria analysis and confirmed in positive mice (5.5 mmol/L) by blood glycemia measurement. Mice were considered diabetic when blood glycemia was superior to 180 mg/dL during two consecutive weeks for HO-1 transgenic NOD models or two consecutive days for Rip-OVAhigh model.

Flow Cytometry: Single-cells suspensions of spleen and lymph nodes were stained with anti-CD8 (clone 53-6.7), anti-CD45.1 (clone A20), anti-CD49b/α2 (clone DX5), anti-CD49d/α4 (clone RI-2), anti-ITGAE/αE (clone M290), anti-CD29/β1-integrin (clone Ha2/5), anti-CD18/β2-integrin (clone C71/16), anti-CD61/β3-integrin (clone 2C9.G2), anti-CD62E (clone 10E9.6), anti-CD62L (clone Mel14), anti-PSGL1 (clone 2PH1), anti-ICAM-1 (clone 3E2), anti-VCAM-1 (clone 429), anti-CD183/CXCR3 (clone CXCR3-173), anti-CD195/CCR5 (clone C34-3448), anti-GITR (clone DTA-1), anti-PD-1 (clone J43), anti-CD28 (clone 37.51), anti-CD38 (clone 90), anti-CD69 (clone H1.2F3), anti-CD44 (clone IM7), anti-CD107b (clone ABL-93) antibodies. All antibodies are from BD Biosciences. Intracellular staining of HO-1 using BD cytofix/cytoperm kit following manufacturers' recommendations were performed with anti-HO-1 antibody (clone HO-1-2, abcam) as primary labeling reagent and with anti-anti-IgG1 (clone MOPC-21) monoclonal antibody conjugated to a fluorochrome as secondary labeling reagent.

Isotype antibodies (Immunotech) were used as a negative control. Staining was assessed using a FACScanto flow cytometer and Diva 6.1 software (Becton Dickinson).

In vivo cytotoxicity assay: Spleen cells from C57/B16 mice were pulsed or not with 5 μM of H2-Kb Ovalbumin (257-264) peptide for 30 min at 37° C. and incubated 5 min at RT in PBS containing 5 and 0.5 μM CFSE respectively. For in vivo cytotoxic test, 3×10⁶ of OVA (257-264) peptide and unpulsed splenocytes were co-injected i.v. into recipient mice. Mice were killed 12 h later, and spleen cells were analyzed by flow cytometry. Cytolytic activity was determined by calculating the percentage of specific lysis using the following formula: 100-([(% Ovalbumin (257-264)-pulsed splenocytes/% unpulsed splenocytes) with autoreactive CTLs/(% Ovalbumin (257-264)-pulsed splenocytes/% unpulsed splenocytes) without autoreactive CTLs]×100).

Histological analysis: For evaluation of insulitis, pancreata were snap-frozen and cryosections (8 μm thick) were acetone-fixed. Sections were stained with H&E (Thermo Electron Corp.), and the degree of insulitis was evaluated microscopically.

Light-sheet-based fluorescent microscopy analysis: Animals were perfused with PFA 4% and washed with PBS. Excised pancreases were clarified using the 3Disco method (40, 41). Briefly, they were dehydrated at room temperature in successive bathes from 50% TetraHydroFuran, anhydrous (THF, Sigma, Saint Quentin Falavier, France) (vol/vol) overnight, 80% THF (vol/vol) for 2 hours to 100% THF two times 1 hour. Then the organs are incubated in a solution of 100% Di-ChloroMethan (DCM, Sigma, Saint Quentin Falavier, France) for 45 minutes and transferred overnight into in the clearing medium 100% DiBenzylEther 98% (DBE, Sigma, Saint Quentin Falavier, France). To image whole adult mice pancreas, we use a home-made light-sheet ultramicroscope (40). The specimen was placed into a cubic cuvette filed with DBE placed on the Z-stage of the bench. It was illuminated with planar sheets of light, formed by cylinder lenses. The light coming from a multi-wavelength (405, 488, 561, 635 nm) laser bench (LBX-4C, Oxxius, Lannion, France) was coupled via two single mode optical fibers into the setup, allowing illumination from one or two sides. Illumination intensity using the 488 nm laser excitation was 10 to 40 mW per light sheet. We used two-sided illumination. The specimen was imaged from above with a MVX10 macroscope, through a PlanApo 1X/0.25 NA or 2X/0.5 NA objective (Olympus, Rungis, France), which was oriented perpendicular to the 488nm light sheet. For GFP fluorescence imaging, we used a 525/50 band pass filter on the turret. Images were captured using a CCD Camera (ORCA AG, Hamamatsu, France) synchronized with the z-stage moving the sample through the light sheet. The home-made ultramicroscope is managed by Micro-manager software and z-stacks of images were taken every 20, 10 or 5 microns depending on the sample and magnification. The images stacks are analyzed using ImageJ software (Rasband, W. S., ImageJ, U. S. National Institutes of Health, Bethesda, Md., USA, http://imagej.nih.gov/ij/, 1997-2012).

Velocity assay: Autoreactive CTLs were cocultured overnight with MHC-II⁺ cells isolated from DLNs of OVA or CoPP-OVA immunized mice. After magnetic depletion of MHC-II⁺cells, re-isolated CTL were settled on a confluent SVEC cells monolayer seeded on ibidi μslide. Live cell imaging was performed at 37° C. using DMI 6000 B microscope (Leica Microsystems, Nanterre, France) under 20× magnification. Images were acquired every 15 s for 30 min with a Cool Snap HQ2 camera (Roper, Tucson, Ariz.) and analyzed with Metafluor 7.1 imaging software (Universal Imaging, Downington, Pa.). The images were imported into the ImageJ software program, and manual cell tracking was performed using the MTrackJ plug-in (developed by Erik Meijering at the University Medical Center Rotterdam, Rotterdam, The Netherlands;http://www.imagescience.org/meijering/software/mtrackj/). The average velocity was calculated for each cell by dividing the total distance traveled by the cell during the last ten minutes.

Transwell migration assay: Autoreactive CTLs were cocultured with MHC-II⁺ cells isolated from DLNs of OVA or CoPP-OVA immunized mice. After magnetic depletion of MHC-II+ cells, re-isolated CTL were deposited on the upper chamber containing a polycarbonate transwell membrane filter (5-μm pore size; Corning). The lower chamber contained 100 ng/ml CXCL12, 100 ng/ml CCL19, or 80 ng/ml CCL17 in DMEM complete medium. The recovered cells after 2 hours at 37° C. were analyzed by flow cytometry.

Cytokine measurement: MHC-II⁺ mice cells or PMA-activated THP-1 cells were treated with CoPP or hemin as described for mouse and cultured for 24 hours with LPS at 1 μg/mL. Supernatants were serially diluted, and cytokine concentration assessed in duplicate by enzyme-linked immunosorbent assay (ELISA). Mouse ELISA kits for IL-12 and IL-10, as well as human IL-1β ELISA kits (BD) were used.

Statistics: For diabetes incidence, significance was calculated using the log-rank test. For all other parameters, significance was calculated by t-paired test, Mann-Whitney nonparametric t-test or one-way anova using Prism software: *p<0.05, **p<0.01, and ***p<0.001.

Results

Genetic and pharmacological manipulation of HO-1 expression prevents T1D: Since DCs play a critical role in T1D onset (19), and because HO-1 exerts its immunosuppressive effect mainly via APCs (17, 18), here we investigated whether HO-1 expression was impaired in DCs from NOD mice. Indeed, we found that compared to their non-diabetic counterparts (NOR mice) (20), female NOD mice exhibited a lower percentage of HO-1-expressing cells among CD11c⁺ cells in the spleen.

To further determine whether HO-1 expression in APCs could impact diabetes, we used pIi-TTA mice, a strain in which the Tet ON system is under the control of the MHC-II invariant chain (Eα-Ii) promoter (21). pIi-TTA mice were crossed with TetO-HO-1 transgenic NOD mice, in which the HO gene is under the control of the hybrid CMV-Tet operator. In the resultant pIi-TTA/TetO-HO-1 double-transgenic mice, doxycycline administration induced dose-dependent increases in HO-1 expression in both bone-marrow derived dendritic cells (BMDCs) and splenic DCs. In female NOD mice, doxycycline-induced HO-1 expression was mainly observed in DCs, and restored HO-1 levels to those of NOR mice.

When doxycycline was provided in drinking water from four weeks of age, the doxycycline-treated pIi-TTA/Tet-O-HO-1 female mice exhibited lower T1D incidence compared to treated single transgenic or untreated littermates. Among doxycycline-treated animals, pIi-TTA/Tet-O-HO-1 mice exhibited reduced leukocyte infiltration compared to Tet-O-HO-1 mice. This phenomenon was dose-dependent. Altogether, our data demonstrated that HO-1 overexpression in MHC-II⁺ APCs reduced T1D incidence in female NOD mice.

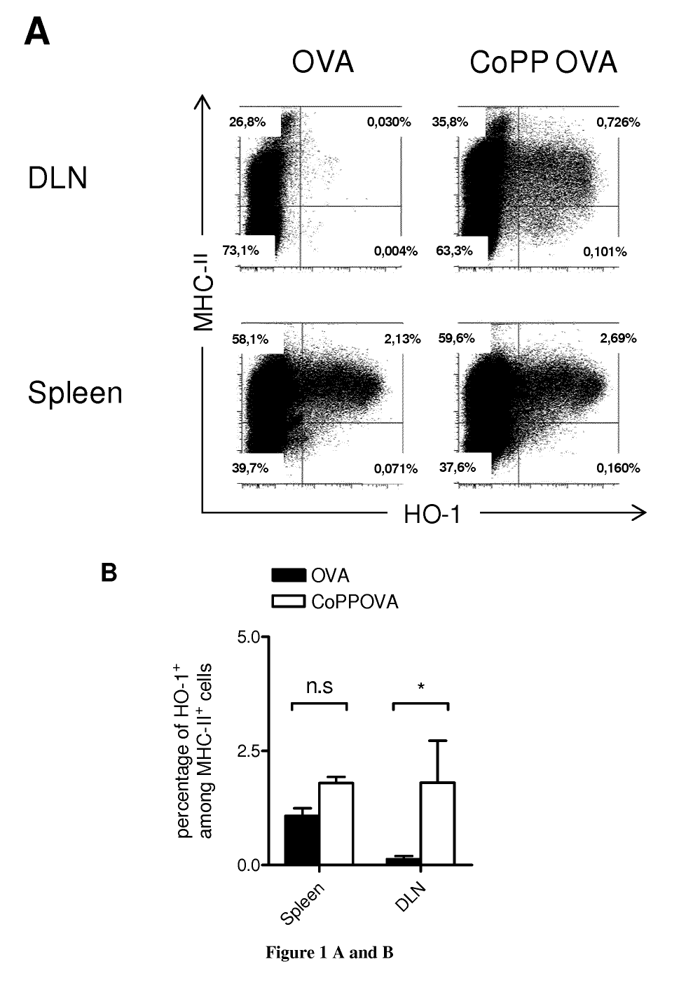

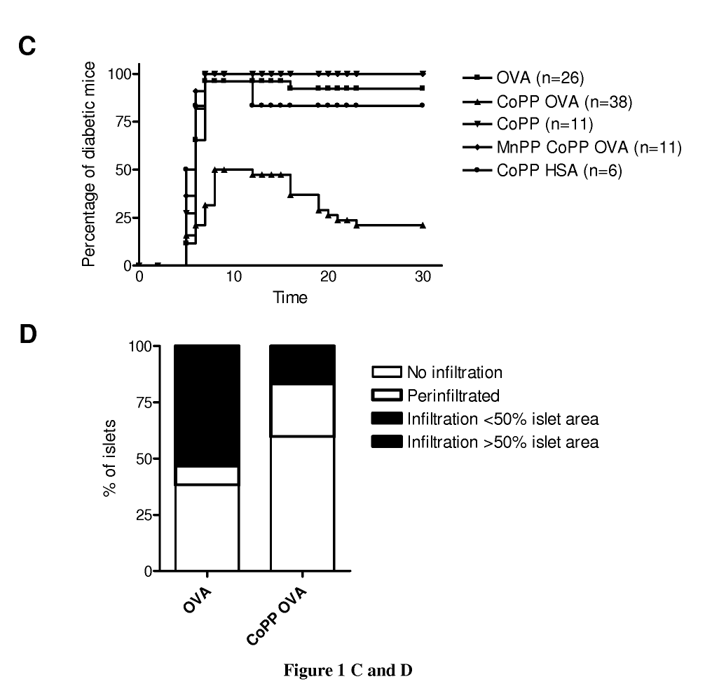

HO-1 inducers inhibit autoreactive CTL-mediated damage: We next investigated whether T1D could be prevented by HO-1 inducers, such as cobalt protoporphyrin (CoPP). For these experiments, we used RIP-OVA^{high} transgenic mice in which OVA is selectively expressed in pancreatic B cells (22). Intradermal injection of CoPP increased HO-1 expression (5.8 fold±1.8) in MHC-II⁺cells in draining lymph nodes (LNs) (FIG. 1A and B). This induction was restricted to the draining LNs, was observed as early as 24 hours after CoPP injection, and lasted for at least 72 hours.

When adoptively transferred with activated OVA-specific CTLs, RIP-OVA^{high} transgenic mice rapidly developed T1D, as previously reported (22). However, co-injecting RIP-OVA^{high} transgenic mice with OVA and CoPP one day before CTL transfer reduced both diabetes (FIG. 1C) and insulitis as analyzed by immunohistology at day 6-8 (FIG. 1D) and by light-sheet-based fluorescent microscopy at 18 h. Such protection was not observed in mice injected with either OVA or CoPP alone or with CoPP and human albumin (FIG. 1C), suggesting that CoPP mediated this protection by interfering with antigen-specific immune responses. Administration of the HO-1 enzymatic inhibitor MnPP also abolished protection, further implying that this protection relied on increased HO-1 enzymatic activity (FIG. 1C). Interestingly, protection was not achieved when CoPP and OVA were injected i.v. (FIG. 8). Altogether, these results demonstrated that activated antigen-specific CTLs could be tolerized in vivo upon intradermal injection of their cognate antigen together with CoPP.

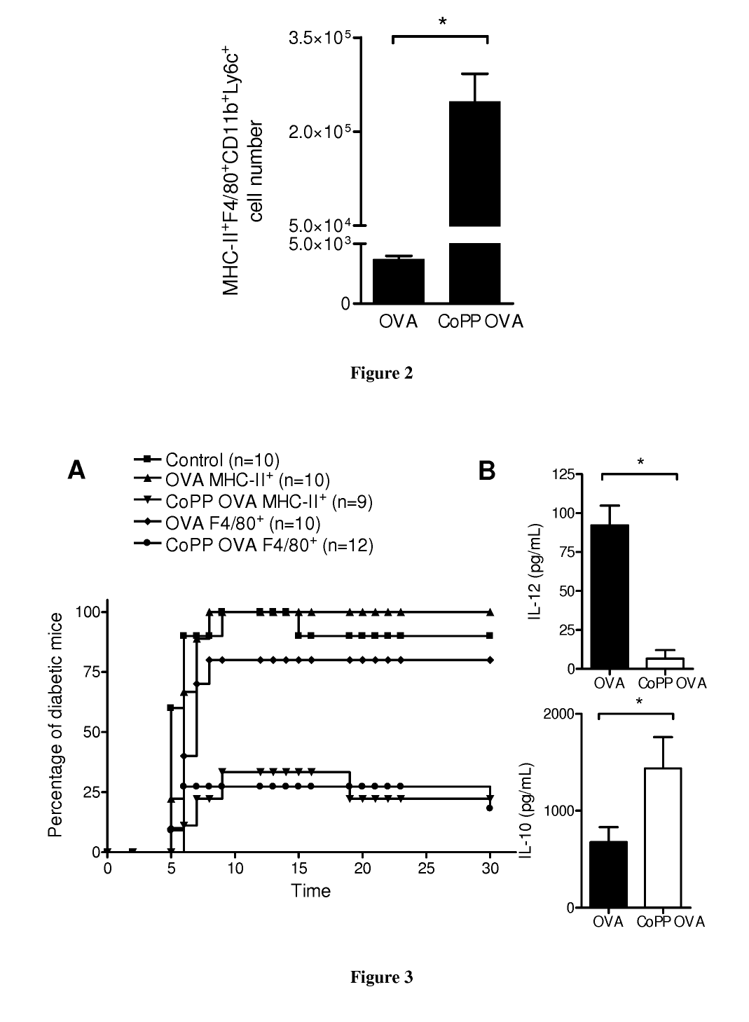

MoDCs tolerize CTL in mice treated with HO-1 inducers: Since co-injection of CoPP and OVA dramatically increased the number of HO-1⁺ MHC-II⁺ cells in draining LNs (FIG. 1A and B), we further characterized these cells for surface molecule expression and for their ability to tolerize preactivated CTLs. Through this, we aimed to elucidate the mechanisms by which CTLs were tolerized in CoPP/OVA-treated mice. Twenty-four hours after intradermal injection of CoPP and OVA, the vast majority of MHC-II⁺HO-1⁺ cells recruited in the draining LNs exhibited a CD11b⁺CD11c^{low}F4/80⁺CD64⁺Ly6C⁺FcεRI⁺ surface phenotype, which is a distinctive feature of Monocyte-derived DCs (MoDCs) (23). Following CoPP/OVA immunization, these cells were massively recruited in the draining LNs in contrast to OVA immunization (FIG. 2). Previous studies have demonstrated that MoDC cannot be recruited in CCR2 deficient mice and, accordingly, the number of HO-1⁺ MoDCs in the LNs of CoPP/OVA immunized mice was dramatically reduced in CCR2-deficient mice (1.5×10³ cells/LN, n=3) compared to in WT animals (2.15×10⁵ cells/LN, n=3), confirming the monocytic origin of the HO-1⁺ APCs in CoPP/OVA immunized mice.

MoDC can be recruited in the draining LN from circulating monocytes present either in the blood or in the dermis (24). Administration of the D₂ prostaglandin receptor agonist BW245c as a dermal migration inhibitor did not affect MoDC recruitment, suggesting that

MoDCs were recruited directly from the blood. This result was confirmed by the extent of MoDC recruitment after excision of the CoPP-injected site one hour after CoPP injection, which was incompatible with a skin origin of the MoDCs.

To further investigate whether HO-1⁺ MoDCS were responsible for T-cell tolerization in CoPP-injected mice, MHC-II⁺ cells were purified from the draining LN of mice immunized with CoPP and OVA, and incubated for four hours with pre-activated OVA-specific CTLs. Next, these cells were adoptively transferred into RIP-OVA^{high} mice, which were then monitored for T1D. CTLs that were incubated with MHC-II⁺ cells from CoPP/OVA-immunized animals exhibited a decreased ability to induce T1D (FIG. 3A).

Similar results were obtained with CTLs incubated with CD11b⁺CD11c^{low}Ly6C^{high}F4/80⁺ cells isolated from the draining LN of CoPP/OVA treated mice, further suggesting that HO-1⁺ MoDCs were responsible for the tolerization of OVA-specific CTLs observed in mice co-injected with CoPP and OVA. Supporting this hypothesis, we also found that F4/80⁺ cells from the draining LN of mice co-injected with CoPP and OVA secreted lower IL-12 levels and higher IL-10 levels in response to LPS (FIG. 3B), and expressed lower surface levels of co-stimulatory molecules compared to cells purified from mice injected with OVA alone. Moreover, HO-1⁺ MoDCs efficiently endocytosed OVA in accordance with the antigen-specific tolerization properties assigned to these cells.

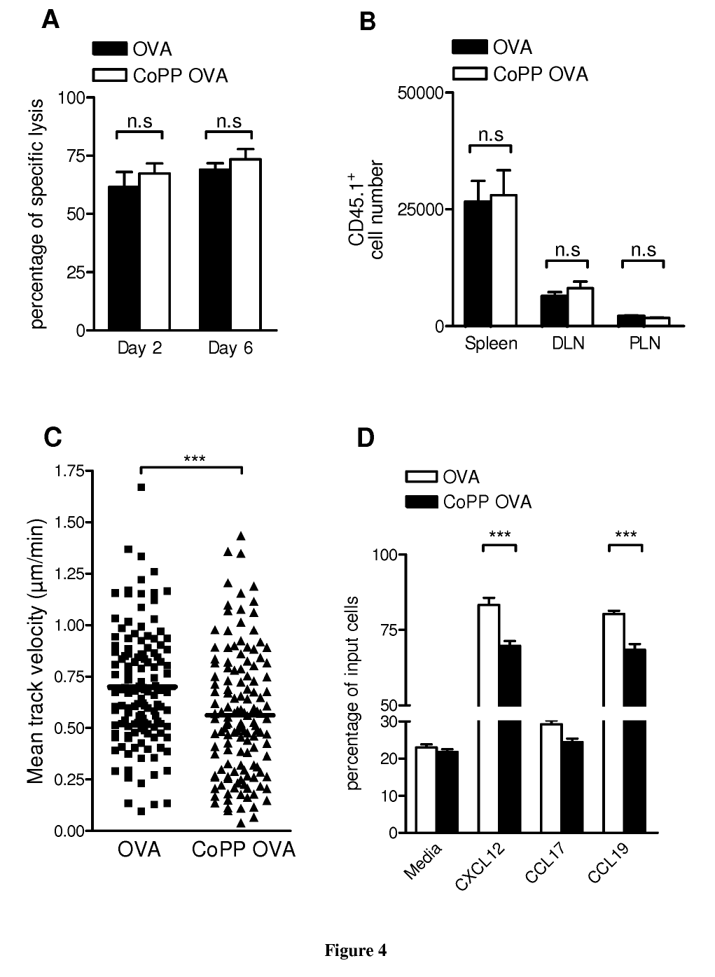

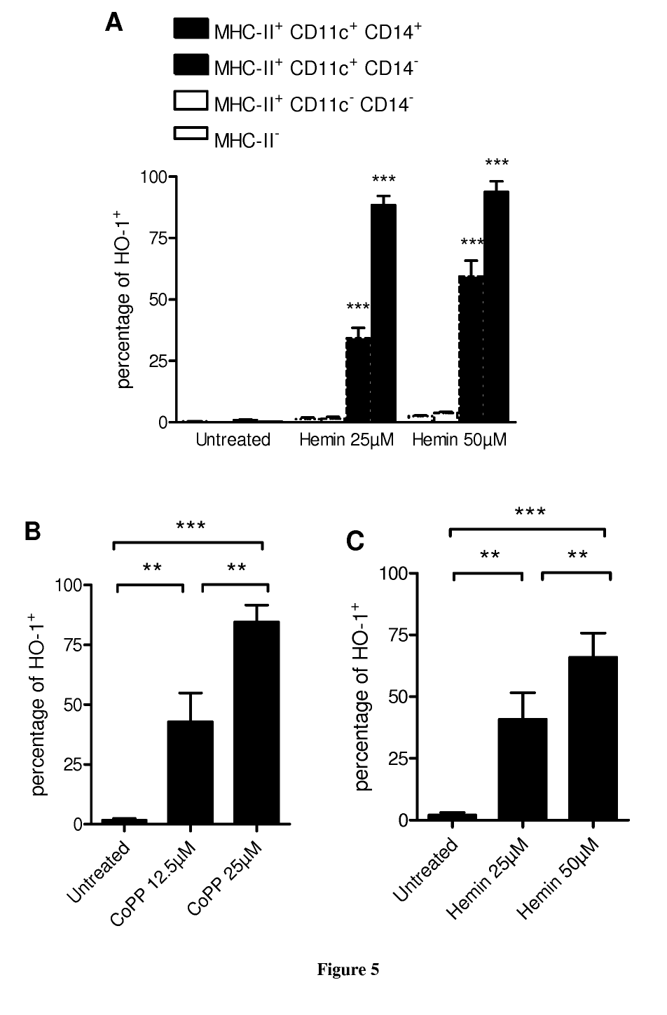

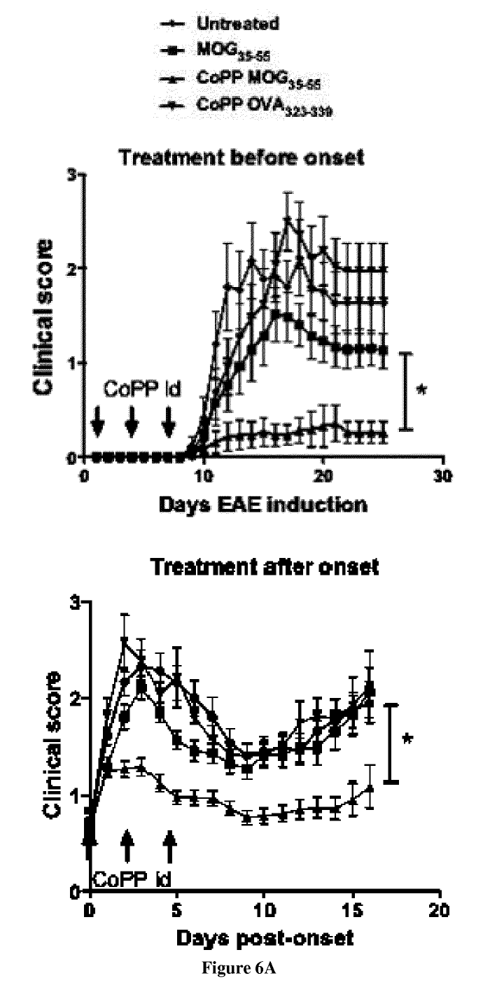

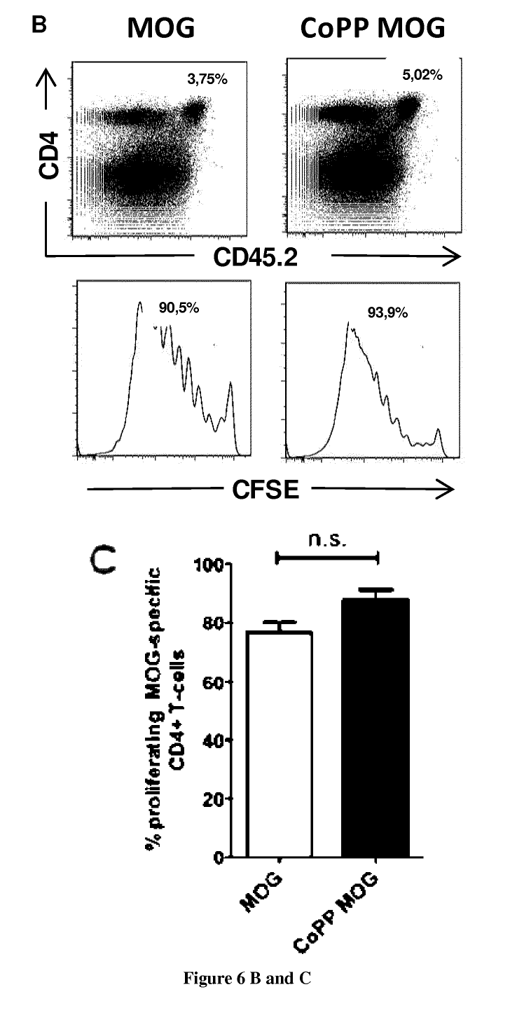

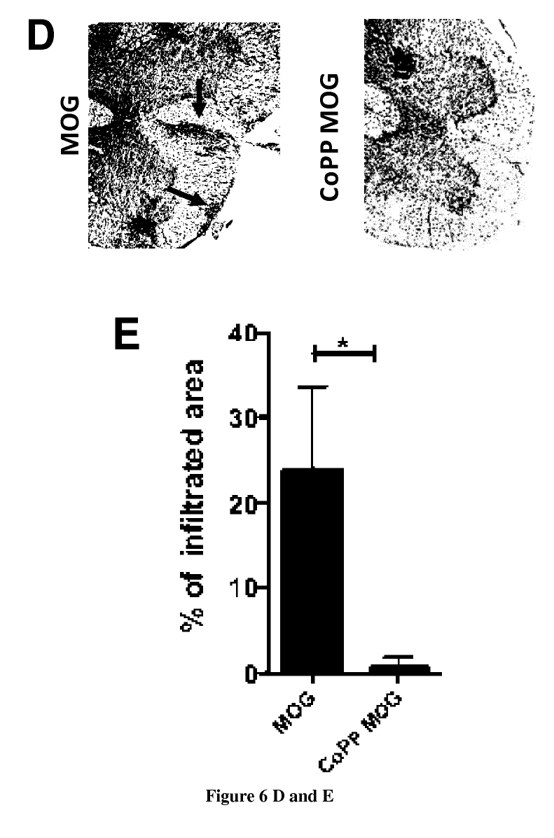

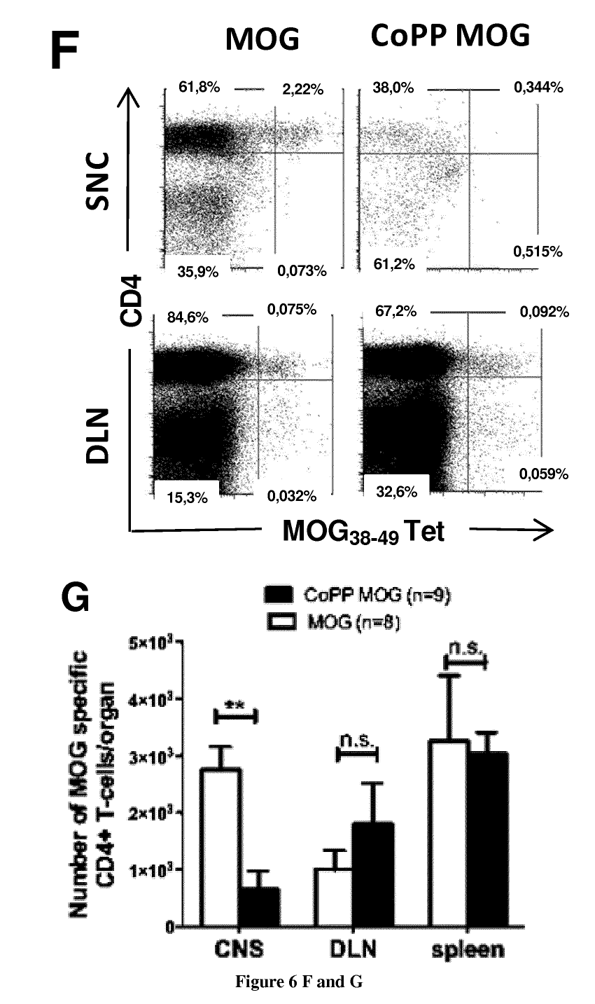

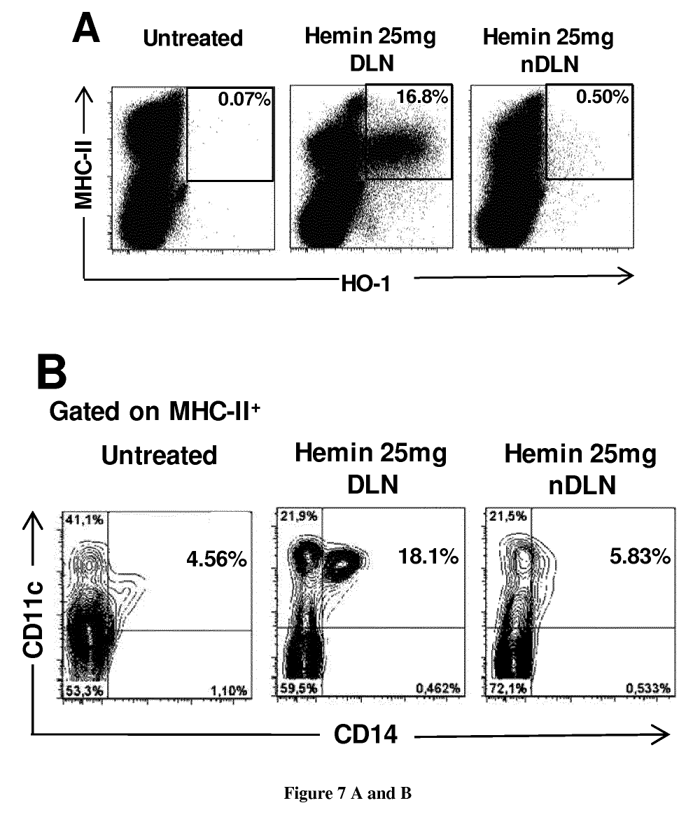

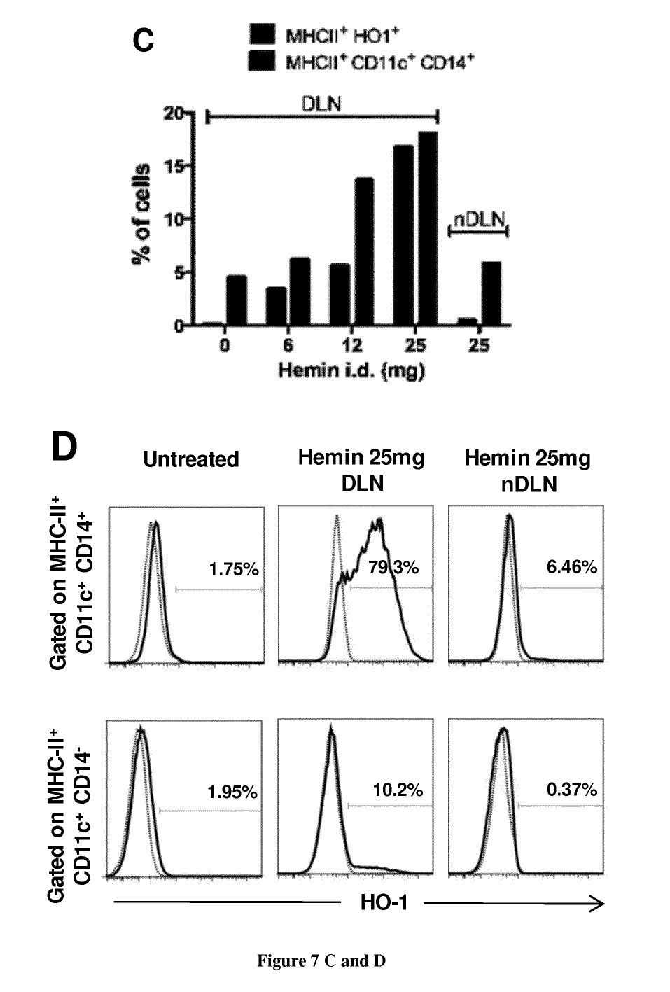

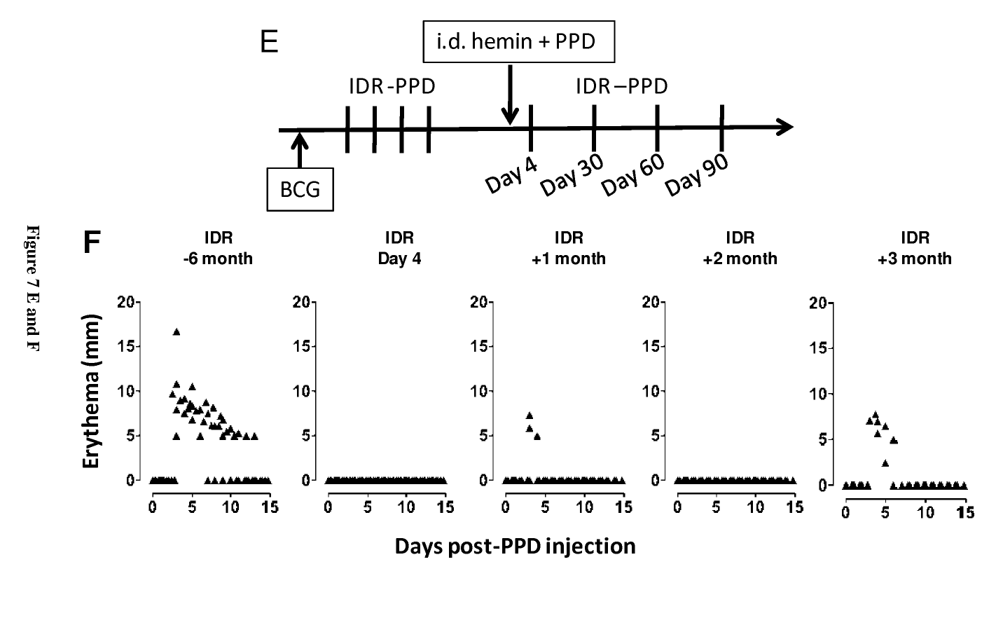

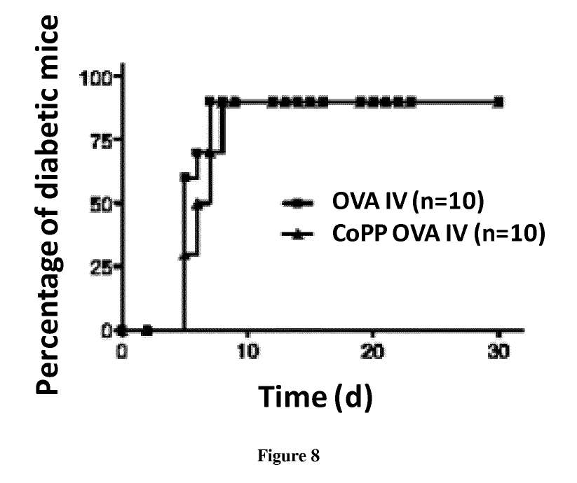

HO-1⁺ MoDCs impair CTL velocity and response to chemokines: We next investigated the mechanism of tolerance of CTL by MoD, to this aim we compared the cytotoxic activities and distributions of OVA-specific CTLs in mice immunized with CoPP/OVA or OVA alone. CTL activities were measured using an in vivo cytolytic assay, and were similar in both groups (FIG. 4A). Furthermore, both groups exhibited similar numbers of OVA-specific CTLs in the spleen, and in draining and pancreatic LNs at two days (FIG. 4B) and six days after injection. In striking contrast, imaging experiments performed 18 hours after T-cell injection showed that OVA-specific CTLs in pancreatic islets were less abundant in CoPP/OVA-immunized mice compared to in OVA-immunized control animals thus suggesting that HO-1⁺ MoDCs altered the ability of CTL to migrate to non-lymphoid tissues.

To further investigate this issue, we performed in vitro experiments measuring these cells' velocity and ability to respond to a chemokine gradient in a transwell migration assay. Compared to non-tolerized control cells, tolerized CTLs exhibited a reduced cell velocity (FIG. 4C) and a decreased ability to migrate along a chemokine gradient (FIG. 4D).

Monitoring the expressions of 20 surface markers involved in CD8⁺ T-cell migration to inflamed tissues, including chemokine receptors and adhesion molecules, revealed that none was differentially expressed in CoPP/OVA- and OVA-immunized mice.

HO-1 inducers selectively recruit MoDCs across species: We next investigated whether a similar phenomenon occurred in humans. With this aim, we incubated PBMCs from healthy human volunteers with hemin. Four hours later, these cells were analyzed for HO-1 expression. As observed in baboons, hemin induced highest HO-1 expression in human MHC-II⁺CD11c^{low}CD14⁺ cells (FIG. 5A). Accordingly, cells from the human monocytic cell line THP-1 dose-dependently expressed HO-1 upon incubation with CoPP (FIG. 5B) or hemin (FIG. 5C). Since THP-1 cells do not express IL-10 and IL-12 upon stimulation we could not compare their expression. However compared to untreated THP-1 cells, HO-1⁺ THP-1 cells secreted reduced levels of the inflammatory cytokine IL-1β and expressed lower surface levels of the co-stimulation markers CD40 and CD86. Furthermore, HO-1⁺ THP-1 cells reduced human CTL velocity. Altogether, these results showed that clinically approved HO-1 inducers selectively target MoDCs across species, and suggested that these cells could tolerize CTLs in both mice and humans.

Discussion

HO-1 induced by chemicals (CoPP) and heme degradation products (e.g., CO) have been shown to inhibit T1D in NOD mice when administered systemically before disease onset (14, 16). However, the mechanisms of action for these treatments have not yet been completely elucidated. Furthermore, these systemic treatments can have secondary effects. In the present paper, we have shown that selective induction of HO-1 in MHC class II-positive cells in 4-week-old mice was sufficient to inhibit T1D in NOD mice. This finding provides a mechanistic explanation for the efficacy of HO-1-based treatment. It is also noteworthy that spleen CD11c⁺ cells from NOD mice expressed lower basal levels of HO-1 than those from their non-diabetic counterpart, NOR mice. This suggests that HO-1, or more likely one of its upstream regulators, could be one of the many genes involved in T1D development in NOD mice. It is possible that inhibition of IL-12 cytokine secretion and increased or maintained IL-10 secretion by HO-1-overexpressing dendritic cells (17, 18) or macrophages (25) may account for these results (26, 27). The explanation may also involve the ability of dendritic cells to tolerize naive anti-β islet CD8⁺ T cells through decreased integrin expression upon exposure to carbon monoxide produced by HO-1 (28).

We reasoned that localized and not systemic HO-1 induction could be used as a tolerogenic strategy for T1D treatment. Parenteral or intranasal vaccination, and oral route administration of self-antigens have been used to induce specific tolerance to β-cell antigens (29). These treatments have occasionally been reported to induce a delay in disease onset (30) or in the decline of C-peptide levels (31), but overall the results have been disappointing.

Notably, the results of such studies have suggested that the antigen and adjuvant selection may be paramount for ensuring the success of this strategy. HO-1 induction confers tolerogenic properties to classical DCs, which inhibit the priming of pathogenic T-cells, as previously demonstrated by our group (18) and others (14).

One of the most striking results in the present study was that MoDCs induced to express HO-1 could inhibit pre-activated CTLs in vitro and in vivo. MoDCs are also called inflammatory DCs because they are recruited in parallel to inflammatory macrophages (23) but contrary to macrophages MoDCs have been shown to cross present efficiently antigens to CD8⁺ cells both in mice and humans. This report provides the first evidence that they can present tolerogenic properties towards activated CD8⁺ T cells. The molecular mechanisms responsible for this phenomenon remain unknown, but several clues are provided in the present results. We found that HO-1⁺ MoDCs secreted high IL-10 levels, and expressed lower levels of costimulatory molecules. This phenotype of HO-1⁺ MoDCs is compatible with a “tolerogenic” function. Indeed, in the natural process of apoptotic erythroid cells engulfment (a process called hemophagocytic) by MoDC lead to both increase HO-1 expression (32) and IL-10 secretion that moderate anti-viral CTL activity (33). Regarding the mechanism of CTLs tolerisation, we found that their proliferation or lytic activity was not impaired following exposure to HO-1⁺ MoDCs in vivo. In striking contrast, tolerized CTLs were impaired in their ability to migrate to non-lymphoid tissues, as demonstrated by their absence in the pancreatic islets of RIP-OVA^{high} mice. This defect was also associated with both a decreased velocity and lower ability to respond to a chemokine gradient in vitro. This finding provides a probable mechanistic explanation for the phenotype of tolerized CTLs.

Representing an improvement over the results of previous strategies using HO-1 or its derivatives as tolerogenic agents to cure autoimmune diseases (14, 16), the tolerance observed following intradermal injection of HO-1 inducer was clinically relevant and antigen-specific. As observed in mice, we found that the intradermal injection of HO-1 inducers in non-human primates resulted in the appearance of HO-1⁺ MoDCs in draining LNs. Most importantly, cells from a human monocyte cell line that were induced to express HO-1 upon incubation with HO-1 inducers show reduced velocity of two different human CTL clones, further suggesting that the same tolerizing mechanisms occur across species. We believe that this is an important finding because HO-1 inducers such as Normosang® and Panhematin® have been already approved for the treatment of acute porphyria in humans (12), therefore paving the way for the use of this molecule to prevent the development of T1D in humans.

### Example 2: Tolerization of Ongoing CTL Response in Experimental Autoimmune Encephalomyetis (EAE)

Material & Methods

Animals: C57BL/6 mice were maintained under safety condition approved by the Inserm and European Union Guidelines. Mice were used between 6 and 10 weeks of age.

Experimental Autoimmune Encephalomyelitis (EAE) induction: Briefly, C57BL/6 were immunized by a subcutaneous injection of emulsified complete freund adjuvant (CFA, sigma aldrich) complemented with mycobacterium tuberculosis (400 μg, BD) and MOG35-55 peptide (200 μg, genecust). Pertussis toxin is injected (200 ng i.v, VWR) at the time of immunization and two days later. Clinical signs of EAE were evaluated daily and scored as follows: 0, normal; 1, limp tail; 2, partial paralysis of the hind limbs; 3, complete paralysis of the hind limbs; 4, hind-limb paralysis and forelimb weakness; 5, moribund or deceased

Treatments: For prophylactic treatment, mice were treated at the time of immunization with one injection in each ear of: CoPP alone (70 μg) or CoPP (70 μg) and MOG peptide (20 μg) or MOG peptide alone (20 μg), or CoPP (70 μg) with irrelevant class II peptide OVA (20 μg). Same injections were repeated twice at three days interval. For curative treatment, mice were treated with one injection in each ear at the EAE onset (i.e., mean clinical score, 0.72±0.1) with the same quantity than in prophylactic treatment. Same injections were repeated twice at three days interval after EAE onset.

Results

Given that HO-1 induction have shown some anti-inflammatory properties for CD4+ T-cells in the EAE model (14), we hypothesized that HO-1-mediated protection may exert an antigen-specific action if both HO-1 expression and antigens presentation occurs at the same time in the same APC. When using a low dose of CoPP (0.2 mg/mouse), we found that i.d. injection of CoPP alone is not sufficient to tolerize against EAE in preventive (administration CoPP treatment on day 0, 3 and 6 after MOG immunization) or curative (CoPP administration 3 times, 3 days apart, after the first clinical signs of EAE appeared) settings (FIG. 6A). However when a low dose of CoPP is co-administered intradermally with cognate antigen (MOG₃₅₋₅₅ class II peptide) tolerization of myelin-specific CD4⁺ T-cells occurs preventively or curatively (FIG. 6A). Such protection was not observed in mice injected with MOG₃₅₋₅₅ alone or with CoPP and irrelevant peptide, demonstrating that CoPP-induced protection is antigen-specific (FIG. 6A). Likewise CoPP-induced CTLs tolerisation, MOG-specific CD4⁺ T-cell have the same proliferation capacity (FIG. 6B,C) in CoPP injected mice and untreated mice. This protection was associated with an inhibition of infiltration in the CNS of CoPP MOG treated mice (FIG. 6D,E). We observed a significant decrease of the absolute number of MOG-specific CD4⁺ T-cells in the CNS in CoPP MOG-treated mice compared to control (FIG. 6F,G) for preventive settings, and for curative settings. Interestingly, in peripheral compartment (spleen and lymph node) the number of MOG specific T cell is unchanged suggesting an impairment of MOG-specific CD4⁺ T-cells migration in the CNS.

Altogether, these results demonstrate that ongoing autoreactive CD4⁺ T cells response is tolerized in vivo upon i.d. injection of their cognate autoantigen and CoPP.

### Example 3: Tolerization of Ongoing Th1 Response in Delayed-Type Hypersensitivity (DHG) in Non-Human Primates

Material & Methods

Animals: Non-Human primates: Baboons (Papio anubis, from the CNRS Primatology Center, Rousset, France) were negative for all quarantine tests. Animals were housed at the large animal facility of our laboratory following the recommendations of the Institutional Ethical Guidelines of the Institut National de la Santé Et de la Recherche Médicale, France.

Intradermal immunization with Normosang®: Three Baboons were injected intradermally in the inguinal fold with respectively 6.25 mg (500 μL), 12.5 mg (500 μL) or 25 mg (1 mL) of clinical hemin (Normosang®). A non-treated baboon has served as control.

Inguinal lymph nodes were surgically removed 24 hours after intradermal injection. Single-cell suspensions for flow cytometry analysis were prepared by enzymatic lymph node disaggregation with Collagenase D (Sigma-Aldricht). All experiments were performed under general anaesthesia with Zoletil (Virbac, Carron, France).

For DTH assays, four baboons were injected intradermally in the two inguinal folds with 25 mg plus 2000 UI of tuberculin-purified protein derivative (PPD; Symbiotics Corporation, San Diego, Calif., USA) in 1.2 mL

BCG vaccination and DTH assay: Baboons were immunized intradermally (i.d.) twice with a bacillus Calmette-Guérin (BCG) vaccine (0.1 nil; 2-8×10 5 UFS; Sanofi Pasteur MSD, Lyon, France) in the upper region of the leg, 4 and 2 weeks before the DTH skin test. Intradermal reactions (IDR) were performed in the back with duplicate intradermal injections of two doses (2000 UI or 40 UI) of tuberculin-purified protein derivative (PPD; Symbiotics Corporation, San Diego, Calif., USA) in 0.1 ml in the skin on the right back of the animals. Saline (0.1 ml) was used as a negative control. Dermal responses at the injection sites were measured using a caliper square. The diameter of each indurated erythema was measured by two observers from days 3-8, and were considered positive when >4 mm in diameter. The mean of the reading was recorded. Other IDRs were performed 4 days, one, two and three month after animals received one i.d. injection of hemin (Normosang®) and 2000 UI of tuberculin-purified protein derivative.

Results

To investigate whether HO-1 inducers could induce HO-1⁺ MoDCs in primates, we injected baboons intradermally with clinical-grade hemin (Normosang®), an HO-1-inducer that has been approved for the treatment of acute porphyrias in humans¹⁰. Hemin injection dose-dependently increased the frequency of HO-1⁺ cells in draining LN but not in the contralateral LNs (FIG. 7A,C). These HO-1⁺ cells expressed MHC-II, CD11c, and CD14 (FIG. 7B,C) by contrast to MHC-II⁺, CD11c⁺, and CD14 negative cells (FIG. 7D), further suggesting that they were MoDCs.

As a first step to investigate whether HO-1 inducers could be used to induce antigen-specific tolerance in primates, we used a delayed type hypersensitivity (DTH) model in baboons. Animals were immunized with BCG vaccine and challenged three consecutive times over five months period with tuberculin intradermal reaction (IDR) (FIG. 7E). These animals showed a measurable and reproducible erythema (minimal size of 4 mm) lasting from 3 to 7 days after injection of tuberculin purified protein derivative (PPD) (FIG. 7F). Six month after the last IDR, animals were tolerized by i.d. injection of Normosang and tuberculin. Four days, one, two and three month after tolerization, IDR was assessed. A strong reduction of IDR was observed following tolerization protocol with a progressive reappearance at three month (FIG. 7F).

As conclusion, i.d. administration of clinical hemin and tuberculin in BCG vaccinated baboons resulted in suppression of T cell memory response against tuberculin for at least two months.

### REFERENCES

Throughout this application, various references describe the state of the art to which this invention pertains. The disclosures of these references are hereby incorporated by reference into the present disclosure.
1. Tsai S, Shameli A, and Santamaria P. CD8+ T cells in type 1 diabetes. Adv Immunol. 2008; 100(79-124.
2. Blancou P, Mallone R, Martinuzzi E, Severe S, Pogu S, Novelli G, Bruno G, Charbonnel B, Dolz M, Chaillous L, et al. Immunization of HLA class I transgenic mice identifies autoantigenic epitopes eliciting dominant responses in type 1 diabetes patients. J Immunol. 2007; 178(11):7458-66.
3. Ouyang Q, Standifer N E, Qin H, Gottlieb P, Verchere C B, Nepom G T, Tan R, and Panagiotopoulos C. Recognition of HLA class I-restricted beta-cell epitopes in type 1 diabetes. Diabetes. 2006; 55(11):3068-74.
4. Panina-Bordignon P, Lang R, van Endert P M, Benazzi E, Felix A M, Pastore R M, Spinas G A, and Sinigaglia F. Cytotoxic T cells specific for glutamic acid decarboxylase in autoimmune diabetes. J Exp Med. 1995; 181(5):1923-7.
5. Pinkse G G, Tysma O H, Bergen C A, Kester M G, Ossendorp F, van Veelen P A, Keymeulen B, Pipeleers D, Drijfhout J W, and Roep B O. Autoreactive CD8 T cells associated with beta cell destruction in type 1 diabetes. Proc Natl Acad Sci U S A. 2005; 102(51):18425-30.
6. Toma A, Laika T, Haddouk S, Luce S, Briand J P, Camoin L, Connan F, Lambert M, Caillat-Zucman S, Carel J C, et al. Recognition of human proinsulin leader sequence by class I-restricted T-cells in HLA-A*0201 transgenic mice and in human type 1 diabetes. Diabetes. 2009; 58(2):394-402.
7. Bluestone J A, Herold K, and Eisenbarth G. Genetics, pathogenesis and clinical interventions in type 1 diabetes. Nature. 2010; 464(7293):1293-300.
8. Keymeulen B, Vandemeulebroucke E, Ziegler A G, Mathieu C, Kaufman L, Hale G, Gorus F, Goldman M, Walter M, Candon S, et al. Insulin needs after CD3-antibody therapy in new-onset type 1 diabetes. N Engl J Med. 2005; 352(25):2598-608.
9. Duarte E A, Eberl G, and Corradin G. Specific tolerization of active cytolytic T lymphocyte responses in vivo with soluble peptides. Cell Immunol. 1996; 169(1):16-23.
10. Kyburz D, Aichele P, Speiser D E, Hengartner H, Zinkernagel R M, and Pircher H. T cell immunity after a viral infection versus T cell tolerance induced by soluble viral peptides. Eur J Immunol. 1993; 23(8):1956-62.
11. Toes R E, van der Voort E I, Schoenberger S P, Drijfhout J W, van Bloois L, Storm G, Kast W M, Offringa R, and Melief C J. Enhancement of tumor outgrowth through CTL tolerization after peptide vaccination is avoided by peptide presentation on dendritic cells. J Immunol. 1998; 160(9):4449-56.
12. Anderson K E, Bloomer J R, Bonkovsky H L, Kushner J P, Pierach C A, Pimstone N R, and Desnick R J. Recommendations for the diagnosis and treatment of the acute porphyrias. Ann Intern Med 2005; 142(6):439-50.
13. Blancou P, Tardif V, Simon T, Remy S, Carreno L, Kalergis A, and Anegon I. Immunoregulatory properties of heme oxygenase-1. Methods Mol Biol. 2011; 677(247-68.
14. Chora A A, Fontoura P, Cunha A, Pais T F, Cardoso S, Ho P P, Lee L Y, Sobel R A, Steinman L, and Soares MP. Heme oxygenase-1 and carbon monoxide suppress autoimmune neuroinflammation. J Clin Invest. 2007; 117(2):438-47.
15. Hu C M, Lin H H, Chiang M T, Chang P F, and Chau L Y. Systemic expression of heme oxygenase-1 ameliorates type 1 diabetes in NOD mice. Diabetes. 2007; 56(5):1240-7.
16. Li M, Peterson S, Husney D, Inaba M, Guo K, Kappas A, Ikehara S, and Abraham N G. Long-lasting expression of HO-1 delays progression of type I diabetes in NOD mice. Cell Cycle. 2007; 6(5):567-71.
17. Chauveau C, Remy S, Royer P J, Hill M, Tanguy-Royer S, Hubert F X, Tesson L, Brion R, Beriou G, Gregoire M, et al. Heme oxygenase-1 expression inhibits dendritic cell maturation and proinflammatory function but conserves IL-10 expression. Blood. 2005; 106(5):1694-702.
18. Remy S, Blancou P, Tesson L, Tardif V, Brion R, Royer P J, Motterlini R, Foresti R, Painchaut M, Pogu S, et al. Carbon monoxide inhibits TLR-induced dendritic cell immunogenicity. J Immunol. 2009; 182(4):1877-84.
19. Jansen A, Homo-Delarche F, Hooijkaas H, Leenen P J, Dardenne M, and Drexhage H A. Immunohistochemical characterization of monocytes-macrophages and dendritic cells involved in the initiation of the insulitis and beta-cell destruction in NOD mice. Diabetes. 1994; 43(5):667-75.
20. Prochazka M, Serreze D V, Frankel W N, and Leiter E H. NOR/Lt mice: MHC-matched diabetes-resistant control strain for NOD mice. Diabetes. 1992; 41(1):98-106.
21. Witherden D, van Oers N, Waltzinger C, Weiss A, Benoist C, and Mathis D. Tetracycline-controllable selection of CD4(+) T cells: half-life and survival signals in the absence of major histocompatibility complex class II molecules. J Exp Med. 2000; 191(2):355-64.
22. Parish I A, Waithman J, Davey G M, Belz G T, Mintern J D, Kurts C, Sutherland R M, Carbone F R, and Heath W R. Tissue destruction caused by cytotoxic T lymphocytes induces deletional tolerance. Proc Natl Acad Sci USA. 2009; 106(10):3901-6.
23. Segura E, and Amigorena S. Inflammatory dendritic cells in mice and humans. Trends Immunol. 2013; 34(9):440-5.
24. Plantinga M, Guilliams M. Vanheerswynghels M, Deswarte K, Branco-Madeira F, Toussaint W, Vanhoutte L, Neyt K, Killeen N, Malissen B, et al. Conventional and monocyte-derived CD11b(+) dendritic cells initiate and maintain T helper 2 cell-mediated immunity to house dust mite allergen. Immunity. 2013; 38(2):322-35.
25. Sierra-Filardi E, Vega M A, Sanchez-Mateos P, Corbi A L, and Puig-Kroger A. Heme Oxygenase-1 expression in M-CSF-polarized M2 macrophages contributes to LPS-induced IL-10 release. Immunobiology. 2010; 215(9-10):788-95.
26. Fujihira K, Nagata M, Moriyama H, Yasuda H, Arisawa K, Nakayama M, Maeda S, Kasuga M, Okumura K, Yagita H, et al. Suppression and acceleration of autoimmune diabetes by neutralization of endogenous interleukin-12 in NOD mice. Diabetes. 2000; 49(12):1998-2006.
27. Lee M S, Mueller R, Wicker L S, Peterson L B, and Sarvetnick N. IL-10 is necessary and sufficient for autoimmune diabetes in conjunction with NOD MHC homozygosity. J Exp Med. 1996; 183(6):2663-8.
28. Simon T, Pogu S, Tardif V, Rigaud K, Remy S, Piaggio E, Bach J M, Anegon I, and Blancou P. Carbon monoxide-treated dendritic cells decrease betal-integrin induction on CD8(+) T cells and protect from type 1 diabetes. Eur J Immunol. 2013; 43(1):209-18.
29. von Herrath M, Peakman M, and Roep B. Progress in immune-based therapies for type 1 diabetes. Clin Exp Immunol. 2013; 172(2):186-202.
30. Skyler J S, Krischer J P, Wolfsdorf J, Cowie C, Palmer J P, Greenbaum C, Cuthbertson D, Rafkin-Mervis L E, Chase H P, and Leschek E. Effects of oral insulin in relatives of patients with type 1 diabetes: The Diabetes Prevention Trial—Type 1. Diabetes Care. 2005; 28(5):1068-76.
31. Ludvigsson J, Faresjo M, Hjorth M, Axelsson S, Cheramy M, Pihl M, Vaarala O, Forsander G, Ivarsson S, Johansson C, et al. GAD treatment and insulin secretion in recent-onset type 1 diabetes. N Engl J Med 2008; 359(18):1909-20.
32. Schaer D J, Schaer C A, Schoedon G, Imhof A, and Kurrer M O. Hemophagocytic macrophages constitute a major compartment of heme oxygenase expression in sepsis. Eur J Haematol. 2006; 77(5):432-6.
33. Ohyagi H, Onai N, Sato T, Yotsumoto S, Liu J, Akiba H, Yagita H, Atarashi K, Honda K, Roers A, et al. Monocyte-derived dendritic cells perform hemophagocytosis to fine-tune excessive immune responses. Immunity. 2013; 39(3):584-98.
34. Vizler C, Bercovici N, Heurtier A, Pardigon N, Goude K, Bailly K, Combadiere C, and Liblau R S. Relative diabetogenic properties of islet-specific Tc1 and Tc2 cells in immunocompetent hosts. J Immunol. 2000; 165(11):6314-21.
35. Hogquist K A, Jameson S C, Heath W R, Howard J L, Bevan M J, and Carbone F R. T cell receptor antagonist peptides induce positive selection. Cell. 1994; 76(1):17-27.
36. Auwerx J. The human leukemia cell line, THP-1: a multifacetted model for the study of monocyte-macrophage differentiation. Experientia. 1991; 47(1):22-31.
37. Roulois D, Vignard V, Gueugnon F, Labarriere N, Gregoire M, and Fonteneau J F. Recognition of pleural mesothelioma by mucin-1(950-958)/human leukocyte antigen A*0201-specific CD8+ T-cells. Eur Respir J. 2011; 38(5):1117-26.
38. Guillerme J B, Boisgerault N, Roulois D, Menager J, Combredet C, Tangy F, Fonteneau J F, and Gregoire M. Measles virus vaccine-infected tumor cells induce tumor antigen cross-presentation by human plasmacytoid dendritic cells. Clin Cancer Res. 2013; 19(5):H47-58.
39. Morgan D J, Liblau R, Scott B, Fleck S, McDevitt H O, Sarvetnick N, Lo D, and Sherman L A. CD8(+) T cell-mediated spontaneous diabetes in neonatal mice. J Immunol. 1996; 157(3):978-83.
40. Dodt H U, Leischner U, Schierloh A, Jahrling N, Mauch C P, Deininger K, Deussing J M, Eder M, Zieglgansberger W, and Becker K. Ultramicroscopy: three-dimensional visualization of neuronal networks in the whole mouse brain. Nat Methods. 2007; 4(4):331-6.
41. Erturk A, Becker K, Jahrling N, Mauch C P, Hojer C D, Egen J G, Hellal F, Bradke F, Sheng M, and Dodt H U. Three-dimensional imaging of solvent-cleared organs using 3DISCO. Nat Protoc. 2012; 7(11):1983-95.

1. A method for inducing immune tolerance in a patient suffering from or at risk of a condition related to immune tolerance, comprising
administering to the patient a therapeutically effective amount of a composition comprising (i) a Heme Oxygenase-1 (HO-1) inducer and (ii) at least one pathogenic antigen involved in the condition,
wherein the composition is administrated topically or intradermally to a patient's skin of the patient.

2. The method of claim 1, wherein the pathogenic antigen is not insulin.

3. The method of claim 1 wherein the HO-1 inducer is not rapamycin.

4. The method according to claim 2, wherein the HO-1 inducer is Cobalt protoporphyrin (CoPP), protoporphyrin IX containing a ferric iron ion (Heme B) with a chloride ligand (Hemin), hematin, iron protoporphyrin or heme degradation products.

5. The method according to claim 2, wherein said pathogenic antigen is an autoantigen, an alloantigen or an allergen.

6. The method according to claim 5, wherein said autoantigen is selected from the group consisting of myelin-related antigen, myelin oligodendrocyte glycoprotein (MOG) and proteolipid protein (PLP).

7. The method according to claim 5, wherein said autoantigen is selected from the group consisting of glutamic acid decarboxylase 65 (GAD65), glial fibrillary acidic protein (GFAP), islet-specific glucose-6-phosphatase catalytic subunit-related protein (IGRP), insulinoma-associated antigen-2 (IA-2) and zinc transporter 8 (ZnT8).

8. The method according to claim 5, wherein said autoantigen is type II collagen (CTII).

9. The method according to claim 5, wherein the alloantigen is selected from the group consisting of antigens expressed by the allograft, proteins expressed in the course of gene therapy and therapeutic proteins.

10. The method according to claim 2, wherein said composition is formulated for subcutaneous, intradermal or topical administration.

11. (canceled)

12. The method of claim 1, wherein the immune tolerance is antigen-specific tolerance.

13. A method for preventing or reducing transplant rejection in a patient in need thereof, comprising a step of
administering to the patient a therapeutically effective amount of a composition comprising (i) a Heme Oxygenase-1 (HO-1) inducer and (ii) at least one pathogenic antigen relevant to the transplant,
wherein the composition is administrated topically or intradermally to skin of the patient,
and wherein the pathogenic antigen is not insulin.

14. A method for preventing or treating an autoimmune disease, an unwanted immune response against proteins expressed in the course of gene therapy or therapeutic proteins, and/or an allergy in a patient in need thereof, comprising
administering to the patient a therapeutically effective amount of a composition comprising (i) a Heme Oxygenase-1 (HO-1) inducer and (ii) at least one pathogenic antigen involved in the autoimmune disease, the unwanted immune response or the allergy,
wherein the composition is administrated topically or intradermally to skin of the patient,
and wherein the pathogenic antigen is not insulin.

15. The method according to claim 14, wherein the autoimmune disease is selected from the group consisting of rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (e.g. Crohn's disease and ulcerative colitis), autoimmune gastric atrophy, pemphigus vulgaris, psoriasis, vitiligo, type 1 diabetes (T1D), non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired haemophilia and thrombotic thrombocytopenic purpura (TTP).

16. (canceled)

17. An in vitro or ex vivo method for generating a population of tolerogenic antigen-presenting cells (APCs) specific for an antigen of interest, comprising a step of culturing a population of APCs with a culture medium comprising a heme oxygenase-1 (HO-1) inducer and said antigen of interest.

18. A population of antigen-specific tolerogenic APCs generated by the method of claim 17.

19. The method of claim 6, wherein the myelin-related antigen is myelin basic protein (MBP) or MBP83-102 peptide.

20. The method of claim 6, wherein the MOG is a MOG35-55 peptide.

21. The method of claim 6, wherein the PLP is a PLP139-151 peptide.
